(19) United States
(12) Patent Application Publication
(10) Pub. No.: US 2021/0272697 A1
(43) Pub. Date: Sep. 2, 2021

(54) Brain and Psychological Determinants of Placebo Response in Patients with Chronic Pain

(71) Applicant: Northwestern University, Evanston, IL (US)

(72) Inventors: A. Vania Apkarian, Chicago, IL (US); Etienne Vachon-Presseau, Montreal (CA); Sara Berger, Evanston, IL (US); Thomas J. Schnitzer, Chicago, IL (US)

(21) Appl. No.: 17/255,911

(22) PCT Filed: Jul. 8, 2019
(86) PCT No.: PCT/US2019/040798

§ 371 Date: Dec. 23, 2020

Related U.S. Application Data
(60) Provisional application No. 62/694,546, filed on Jul. 6, 2018.

(51) Int. Cl. G16H 50/30; G16H 10/20; G16H 30/40

(52) U.S. Cl. CPC G16H 50/30; G16H 30/40; G16H 10/20

(57) ABSTRACT

Methods are disclosed for determining brain and psychological determinants of placebo response in individuals. The method may include creating a biosignature for use as a predictor of placebo response by neuroimaging a brain of an individual with magnetic resonance imaging, identifying a prefrontal cortex functional connection, identifying capacity of awareness and regulation of emotions, and stratifying the individual as a predicted placebo responder or a non-responder based upon the prefrontal cortex functional connection, capacity of awareness, and regulation of emotions. Methods are also disclosed for treating a patient with chronic pain using a biosignature to predict placebo response and wherein the treatment is inert.

### RELATED APPLICATION DATA

This application claims the benefit U.S. Provisional Patent Application No. 62/694,546, filed on Jul. 6, 2018, which is hereby incorporated by reference in its entirety.

### STATEMENT OF GOVERNMENT INTERESTS

This invention was made with government support under NIH/NCCIH R01AT007987 awarded by National Institutes of Health. The government has certain rights in the invention.

### BACKGROUND

The present disclosure relates generally to medical treatment. More specification, the present disclosure relates to methods for determining brain and psychological determinants of placebo responses in individuals.

Placebos have always been integral to the healing practices and medicine. Usually, a placebo is recognized as a legitimate and useful component of medical therapeutics. Generally, a placebo response is universally observed in randomized controlled trials (RCTs), yet these effects are commonly dismissed as consequences of uncontrollable confounds. Powerful placebo effects have been demonstrated in a range of common medical conditions, including peptic ulcers, irritable bowel, hypertension, low back pain, arthritis, anxiety disorders, depression and ADHD.

However, conventional mechanisms to determine placebo effects in patient encounter multiple technical problems. For example, the placebo effect is observed universally in almost all randomized placebo-controlled clinical trials (RCT), particularly in pain treatment trials. The current scientific dogma assumes that RCT-related placebo responses are primarily due to uncontrollable confounds. Therefore, the current state of the art has failed to identify the underlying properties of the placebo effect. Furthermore, the neurobiological mechanisms underlying the placebo effect have been almost exclusively studied for acute responses to conditioning-type manipulations in healthy individuals. However, in healthy subjects, placebo responses and their neural and psychological correlates lack consistency across different routes of administration. Additionally, in clinical chronic pain settings, the translation of such a placebo response is questionable patriating to the fact that (I) chronic pain patients exhibit distinct brain anatomy and physiology, and (II) chronic pain patients are repeatedly exposed to a myriad of medical rituals which may bias expectations toward treatment. Moreover, functional connectivity is malleable and reflects learned associations, given the hypothesis that the RCT placebo effect is embedded in predictable psychology and neurobiology. Furthermore, the current studies into the placebo effect lack a proper treatment arm, and rely on only one cross-sectional measurement of pain. The current studies also do test the within-subject reliability of neural predictor after treatment in patients. Although, the current studies have made various prediction, such predictions may ignore the natural propensity of a patient to respond in a clinical setting and do not test the within-subject reliability of the marker post treatment.

Therefore, in light of the foregoing discussion, there exists a need to overcome the aforementioned drawbacks associated to determine placebo effects in a patient.

### SUMMARY

Disclosed herein are methods for determining brain and psychological determinants of placebo responses in individuals. The present disclosure seeks to provide a solution to the existing problems associated with conventional mechanisms to determine placebo effects in patients. An aim of the present disclosure is to provide a solution that overcomes at least partially the problems encountered in the prior art, and provides an efficient and reliable method for determining brain and psychological determinants of placebo responses in individuals.

In one aspect, an embodiment of the present disclosure provides a method of identifying a placebo responder in an individual or a group of individuals by obtaining an individual or group of individuals as participants; pre-specifying inclusion criteria; conducting a study, wherein the study is conducted in a clinical randomized controlled trial setting for assessing the placebo response, and wherein the study is conducted across at least six visits to the randomized controlled setting; randomizing participant allocation pertaining to the study, wherein the randomization scheme assigns each participant a randomization code and classifies the participants into one of: placebo group, no treatment group, active treatment group; administering treatment to the participants; monitoring pain intensity of the participants by receiving pain ratings from the participants, via a software application, and wherein the software application is executed on a smartphone or a computer connected to the internet; pre-processing the ratings; performing a blind analysis; performing brain imaging and data analysis; analyzing questionnaire data; predicting a magnitude of a placebo response; and administering further treatment to the individual or group of individuals based on brain properties and psychological determinants of the placebo response.

In another aspect, a method of creating a biosignature for use as a predictor of placebo response is disclosed that includes neuroimaging a brain of an individual with magnetic resonance imaging, identifying a prefrontal cortex functional connection, wherein the prefrontal cortex functional connection is a functional coupling of a dorsolateral prefrontal cortex (DLPFC), a periaqueductal grey (PAG), a rostral anterior cingulate cortex (rACC), a precentral gyrus (PreCG), or a combination thereof. In other aspects, the method includes identifying capacity of awareness and regulation of emotions, wherein the capacity of awareness and the regulation of emotions is determined by the individual personality traits, wherein the personality traits are determined by the individual completing a questionnaire, and wherein the personality traits include optimism, anxiety, extraversion, neuroticism or a combination thereof. In still other aspects, the method of creating a biosignature includes stratifying the individual, as a predicted placebo responder of a non-responder, based upon the prefrontal cortex functional connection, capacity of awareness, and regulation of emotions.

In still other aspects, a method of treating a patient with chronic pain is disclosed herein that includes neuroimaging a brain of a patient with magnetic resonance imaging, identifying a prefrontal cortex functional connection, wherein the prefrontal cortex functional connection is a functional coupling of a dorsolateral prefrontal cortex (DLPFC), a periaqueductal grey (PAG), a rostral anterior cingulate cortex (rACC), a precentral gyrus (PreCG), or a combination thereof. In other aspects, the method may include identifying capacity of awareness and regulation of emotions, wherein the capacity of awareness and the regulation of emotions is determined by the individual personality traits, wherein the personality traits are determined by the individual completing a questionnaire, and wherein the personality traits include optimism, anxiety, extraversion, neuroticism or a combination thereof. In yet other aspects, the method may include determining if the patient is a placebo responder based upon the prefrontal cortex functional connection, capacity of awareness, and regulation of emotions, and then treating the patient with chronic pain, wherein the treatment is inert, and wherein the chronic pain includes chronic back pain, osteoarthritis, fibromyalgia, or peripheral neuropathy.

In some embodiments, the disclosed methods may substantially eliminate or at least partially address the aforementioned problems in the prior art, and enable accurate, efficient, and reliable determination of brain and psychological determinants of placebo response in individuals.

Additional aspects, advantages, features and objects of the present disclosure may be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, may be better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those in the art will understand that the drawings are not to scale. Wherever possible, like elements have been indicated by identical numbers.

The patent or application file contains at least one drawing executed in color. Copies of this patent or patent application publication with color drawing(s) will be provided by the Office upon request and payment of the necessary fee. The foregoing and other features and advantages of the present embodiments will be more fully understood from the following detailed description of illustrative embodiments taken in conjunction with the accompanying drawings in which:

FIGS. 1A-1J illustrate methods for testing the placebo effects on patients in a clinical trial and the results of such methods, in accordance with one embodiment of the present disclosure.

FIGS. 2A-2G illustrate the effects of a placebo on the dorsolateral prefrontal cortex network of patients, in accordance with an embodiment of the present disclosure.

FIGS. 3A-3C illustrate the effects of a placebo on the subcortical limbic volume asymmetry and sensorimotor cortex, in accordance with an embodiment of the present disclosure.

FIGS. 4A-4E illustrate personality traits and psychological factors of a patient in response to a placebo, in accordance with an embodiment of the present disclosure.

FIGS. 5A-5K illustrates machine learning for predicting the magnitude of response from prospective data, in accordance with an embodiment of the present disclosure.

FIGS. 6A-6F illustrate and describe a biosignature for placebo response and its validation.

FIGS. 7A-7C illustrate the biosignature predicted placebo response.

FIGS. 8A-8C illustrate examples of pain trajectories observed from a patient showing no improvement or analgesia following the administration of a treatment.

FIG. 9 illustrates results from patients responding to either PTx or MTx and the related connectivity between the DLPFC-PreCG.

### DETAILED DESCRIPTION

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

The disclosed subject matter may be further described using definitions and terminology as follows. The definitions and terminology used herein are for the purpose of describing particular embodiments only, and are not intended to be limiting.

As used in this specification and the claims, the singular forms “a,” “an,” and “the” include plural forms unless the context clearly dictates otherwise. For example, the term “patient” should be interpreted to mean “one or more patients” and unless the context clearly dictates otherwise. As used herein, the term “plurality” means “two or more.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into subranges as discussed above.

A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

In the present disclosure, the terms “patient,” “subject,” and “individual” may refer to a human or non-human being. The terms “patient,” “subject,” and “individual” may be used interchangeably throughout the present disclosure.

Brain and Psychological Determinants of Placebo Response in Patients with Chronic Pain

In one aspect, the present disclosure provides a method of testing, identifying, and/or determining brain and psychological determinants of placebo responses in individuals, characterized in that the method includes one or more of the following steps: (i) obtaining participants; (ii) pre-specifying inclusion criteria; (iii) conducting a study, optionally wherein the study is conducted in a clinical randomized controlled trial setting for assessing the placebo response, optionally wherein the study is conducted across at least six visits to the randomized controlled setting; (iv) randomizing participant allocation pertaining to the study, optionally wherein the randomization scheme assigns each participant a randomization code and classifies the participants into one of: placebo group, no treatment group, active treatment group; (v) administering treatment to the participants; (vi) monitoring pain intensity of the participants by receiving ratings from the participants, optionally via a software application, optionally wherein the software application is executed on a smartphone or a computer connected to the internet; (vii) pre-processing the ratings; (viii) performing blinding of analysis; (ix) performing brain imaging and data analysis; (x) analyzing questionnaire data; (xi) predicting magnitude of a placebo response; (xii) administering further treatment based on the brain and/or psychological determinants of the placebo response.

The present disclosure discloses various embodiments of the aforementioned method for determining brain and psychological determinants of placebo responses in individuals. Beneficially, the placebo response could be efficiently predicted in chronic pain patients based on the study of psychobiology of placebo responses as disclosed herein. The present disclosure combines complex inter-relationships, such as psychological, functional, behavioral, anatomical, computational and models of machine learning, and most importantly, human transformation, interpretation, and modification of the data, to predict the magnitude of a response to a placebo. Beneficially, the treatment outcomes have high level of dimensionality of chronic pain outcomes without neglecting pain quality. Additionally, the treatment is efficacious and robust in providing the unbiased results using placebo-based randomized controlled trials.

Throughout the present disclosure, the term “placebo” used herein refers to a substance or treatment with no active therapeutic effect. A “placebo” may include but is not limited to, a pill or other pharmaceutical form of an active pharmaceutical ingredient, or a placebo may refer to another non-treating therapy. A placebo resembles an active medication or therapy, and influences the response towards a treatment. Optionally, the placebo may be formulated for oral administration. For example, the placebo can be formulated as a tablet (like sugar pills), a capsule, a mini-tablet, a syrup (distilled water), a powder, a sprinkle and so forth. In an embodiment, the placebo can be an inert injection (like saline injection, glucose injection), a sham surgery and so forth. Moreover, the placebo further comprises one or more pharmaceutically acceptable additives, such as a carrier, a binder, a suspending (or a dispersing) agent, a filling agent, a coloring agent, a flavoring agent (such as a sweetener), disintegrating agent, diluting agent, wetting (or moistening) agent, anti-foaming agent, antioxidant, lubricant, solubilizer, plasticizer, stabilizer, preservative or one or more combination thereof. The placebo functions as a control to prevent a recipient(s) and/or others from knowing whether a treatment is active or inactive. More specifically, psychological expectations about efficacy can influence results corresponding to the treatment.

Throughout the present disclosure, the term “placebo effect” or “placebo response” used herein refers to an improvement in symptoms caused by receiving an inert treatment or a response to an inert treatment. The placebo effect varies with different conditions, biological systems, and treatment types. Moreover, the placebo effect is observed universally in almost all randomized controlled (or randomized placebo-controlled) clinical trials (indicated by RCT hereafter), such as pain treatment trials. The placebo effect is usually equivalent or superior to the active treatment and further increases with recent (namely, fresh or new) RCTs. It will be appreciated that the RCT-related placebo effect (or response) may depend on multiple parameters. Optionally, the multiple parameters may include a plurality of biological properties (or features) and other psychological characteristics. More optionally, the plurality of brain properties may include at least one of: a subcortical limbic volume asymmetry, a sensorimotor cortical thickness, a functional coupling of a dorsolateral prefrontal cortex (DLPFC) with a periaqueductal grey (PAG), a rostral anterior cingulate cortex (rACC), a precentral gyrus (PreCG) from resting state functional magnetic resonance imaging (rsfMRI); and the psychological characteristics may include interoceptive awareness, openness, and so forth. Furthermore, all the plurality of brain properties may be observed as being present before exposure to the placebo; moreover, most of the plurality of brain properties may be determined to be stable across treatment and washout periods. However, in an example, the functional coupling between DLPFC and PAG may be determined to shift with repeated exposure and response type. A psychological profile is associated with placebo response in CBP departs from the literature regarding placebo in healthy subjects. None of the often-cited personality traits in placebo literature—optimism, anxiety, extraversion, neuroticism—successfully differentiate placebo responders from non-responders. Instead, placebo response is driven primarily by a combination of a greater openness to experience, increased emotional awareness, decreased worrying about discomfort, augmented capabilities in describing inner experiences, and higher sensitivity to non-painful situations. The placebo response can be predicted from an ability to recognize subtle cues in the body regarding emotional and physical well-being, to remain attentive to these cues and emotions by not ignoring or suppressing them, and to choose to accept these states as opposed to becoming worried or burdened by them. These factors of personality are able to differentiate PTxResp and PTxNonR as well as predict the magnitude of placebo response in new patients. Notably, the psychological factors are sufficient for classifying patients into placebo responders (indicated by “Resp” hereafter) and non-responders (indicated by “NonR” hereafter), and predicting the magnitude of response associated with the placebo responders and the magnitude of response associated with the placebo non-responders. Furthermore, placebo analgesia during clinical trials depends on a combination of plurality of brain properties and specific psychological factors. Therefore, such brain properties and other psychological factors, are tightly coupled with the magnitude of response or magnitude of analgesia.

Throughout the present disclosure, the term “magnitude of response” or “magnitude of analgesia” used herein may refer to the highest difference between baseline and one or more treatment periods (e.g., 2 treatment periods). The magnitude of response may provide a significant improvement of symptoms (essential for stratifying a patient as a placebo responder), while the % analgesia may represent a continuous measure determining the importance of the response. In an embodiment, the magnitude of response may be predicted from a functional network involving connectivity between nodes mainly located in the limbic community, the DLPFC, the orbitofrontal cortex, and the temporo-parietal junction. Placebo analgesia (or insensitivity) may be relevant in the management of chronic pain, since most pharmacological treatments have long-term adverse effects or addictive properties, or show only modest improvements that are insufficient to achieve clinically meaningful amelioration of disability.

In an example, neuroimaging-based RCT is performed in chronic back pain (indicated by “CBP” hereafter), and patients demonstrate that the intensity, but not quality, of pain is diminished with placebo ingestion. The neurobiological mechanisms underlying the placebo effect is studied for acute responses to conditioning-type manipulations in healthy individuals, as performed in a laboratory setting. In healthy subjects, placebo response and its neural and psychological correlates may lack consistency across different routes of administration, such as when a placebo is taken orally, or by intravenous injection, by intramuscular injection or otherwise. Moreover, in clinical chronic pain settings, the translation of such findings may be questionable not only because chronic pain patients exhibit distinct brain anatomy and physiology but also because such patients are repeatedly exposed to a myriad of medical rituals which may bias expectations toward treatment. For example, neuroimaging studies may suggest predictability of the RCT placebo effect based on brain functional properties in different placebo procedures (patch and pill) examined in chronic back pain and knee osteoarthritis conditions. Since functional connectivity is malleable and reflects learned associations, a comprehensive RCT for placebo ingestion (in contrast to no-treatment) may be designed and conducted in CBP patients.

In the disclosed methods, optionally, the participants are selected from at least one of: chronic back pain patients, knee osteoarthritis patients, peripheral neuropathy patients. Patients may be characterized and/or labeled as responders if they show stronger pain reduction between pain ratings entered during baseline and pain ratings entered in at least one of the two treatment periods compared to the expected range of pain reduction generated by the permutations, and non-responders otherwise. This stratification strategy may be justified because Gaussian mixture models performed on the magnitude of placebo response may suggest that patients are best represented by two subpopulations rather than one (supplement AIC BIC). The response rate may indicate that placebo administration increased the frequency of CBP patients showing pain reduction—more patients are classified as responders in the placebo treatment group (PTxResp 24/43: response rate of 55%) compared to the no treatment group (NoTxResp 4/20: response rate of 20%). Importantly, demographics and external factors such as pain intensity during baseline, phone rating compliance, overall treatment compliance, treatment duration, rescue medication usage, and previous medication usage are not related to placebo response.

The method of determining placebo response in chronic low back pain (CBP) patients typically comprises obtaining participants. Optionally the participants are the selected from a group of individuals with chronic back pain (CBP). More optionally, the participants are selected from any one of: a general population, clinical referrals via hospital databases, advertising in community and so forth. Additionally, the participants include individuals with age 18 years or older and a history of lower back pain for at least 6 months. Optionally, the lower back pain is neuropathic, with no evidence of additional co-morbid chronic pain, neurological, or psychiatric conditions.

The method typically comprises designing a study. Optionally, the study is performed for a predefined period of time. More optionally, the total duration of the study is 2, 4, 6, 12, or 15 months. For example, the first patient is seen on Nov. 6, 2014, and the last patient is seen on Feb. 4, 2016. Optionally, the study is conducted in a setting of a clinical randomized controlled trial specifically designed for assessing the placebo response. More optionally, the study comprises: a plurality of visits (such as 6 visits) spread over a part of the total duration of study, for example such as approximately 8 weeks, including a baseline monitoring/screening period and two treatment periods, each followed by a washout period. The design may be set up to track placebo response in time and to test the likelihood of response to multiple administrations of placebo treatment in order to optimize accuracy in the identification placebo response. The overall protocol may include four scanning sessions.

In an example, on Visit 1, participants are screened for eligibility and consent. Following informed consent, a blood sample is drawn (e.g., in order to perform a comprehensive chemistry panel, a complete blood count, and a pregnancy test if applicable), vital signs are taken (e.g., blood pressure, heart rate, respiration rate, height, and weight), and a medical professional is required to complete a physical examination and take a comprehensive pain history. Additionally, participants are asked to complete a battery of questionnaires regarding basic demographics, pain, mood, and personality. Optionally, these self-reported measures are available online via REDCap (Research Electronic Data Capture version 6.5.16, © Vanderbilt University) through a survey link sent to the participant's email address (or a back-up study email if they do not have an email account). Specifically, once submitted, questionnaire answers are finalized in the database and are rendered un-editable by both participants and study staff. In an embodiment, the participants may be allowed to take breaks and walk around the testing room to avoid questionnaire fatigue due to the number of questionnaires administered, although the patients are required to complete all questionnaires at the designated visit. Optionally, any remaining information, including clinical data collected at the visit, is entered manually into the database by study staff. The relevant information is verified via double-data entry by different staff members at a later time. At the end of Visit 1, participants are asked to stop all medication they are currently prescribed for pain control. Optionally, a rescue medication in form of acetaminophen tablets (500 mg each) is provided as a controlled replacement to be used at any time in the study if the pain becomes too intense. At this time, participants are also trained on how to use the electronic pain rating application on either a phone or a computer; if participants do not have access to either, they are provided with a smartphone and data plan for the duration of the study. The baseline rating period starts at the end of Visit 1 and lasts until Visit 2 (approximately two weeks later).

Optionally, pain is monitored electronically using an application designed specifically for the study. Optionally, the pain is monitored at least twice a day. Specifically, such app is used to track patients' pain over time and to query them on their medication usage; it could be accessed using either a smartphone or a website link on a computer. Optionally, the app comprises a VAS scale with sliding bars for rating a current pain level from 0 (no pain) to 10 (worst imaginable). More optionally, the app comprises fields to indicate the participant's assigned ID number, query regarding rescue medication usage at a time, query regarding the study medication, a comments section usable to describe their pain, mood, or medication usage. Participants are instructed to use the app twice a day, once in the morning and once at night. Furthermore, two different components of the placebo response based on daily ratings provided by smart phone technology: the presence/absence of response to treatment (Resp versus NonR) and the magnitude of the response (% analgesia) can be studied by regular use of the smartphone (or computer) app.

Optionally, a pre-processing of the rating data from all participants is performed. More optionally, the pre-processing of the app rating data comprises: receiving rating twice a day, considering only the last rating as indicative of the participant's final assessment of their pain levels (in case multiple ratings are entered within 30 minutes of each other), multiple ratings outside the 30-minute window are taken as valid entries. It may be appreciated that beside pre-processing the app rating data, no other changes are made to the ratings. In other words, in the instances where participants missed ratings, no attempts are made to interpolate or re-sample the data so that the temporal aspects of the ratings are left intact.

Furthermore, submitted ratings may be immediately sent to a secure server and both date- and time-stamped. Rating compliance is assessed by a separate program, which monitored whether the list of currently enrolled patients provides the necessary ratings during the previous day. Optionally, in the case a patient omits (accidentally or deliberately) a rating, staff is alerted via an email. Alternatively, if patients miss more than 2 consecutive ratings (across 24 hours), a member of the study team is required to contact the patient to remind them to use the app. In an embodiment, patients may be discontinued from the study if they do not comply with the daily rating requirements despite repeated contact from the study team. Optionally, to encourage compliance, participants are compensated (e.g., $0.25) for each rating they submit, up to a maximum (e.g. $0.50/day). More optionally, such additional payment is given to them on the last visit of the trial.

The method typically further comprises pre-specifying an inclusion and exclusion criteria. Optionally, the inclusion criteria comprise one or more of: no concomitant pain medications, use of a smartphone or a computer to monitor pain, frequency of monitoring the pain (such as twice a day), setting a healthy range for monitoring the pain and so forth. Optionally, the participants have at least one of: an age more than or equal to 18 years, a history of lower back pain for at least 6 months, not been prescribed any concomitant pain medications, passed the MRI safety screening requirements at each scanning visit, an access to use a smartphone or a computer. For example, the participants with a pain level of at least 5/10 during the screening interview, and an averaged pain level from the smartphone app equal to higher than 4/10 during the baseline rating period are selected obtain a treatment group. The non-compliance with the pre-specified inclusion criteria forms the exclusion criteria. Optionally, measurements taken at Visit 1 are required to be within the pre-specified healthy range and all participants are required to pass the MRI safety screening requirements at each scanning visit. Furthermore, all participants' ratings are closely monitored for the first two weeks of the study as part of a run-in/baseline pain period. Individuals not meeting this level are deemed ineligible and did not continue in the study (n=16 screen failures). However, it is later noticed that 3 additional participants had met the exclusion criteria but accidentally continued in the study. Specifically, one person is assigned to no-treatment and is discontinued as a protocol deviation before study completion; the other two individuals finished the study in the placebo treatment group but are not included in the analysis.

In an embodiment, Visit 2 is scheduled for patients whose pain ratings and blood lab results meet inclusion criteria. Optionally, the Visit 2 comprises a 35-minute brain imaging session that collects a T1-weighted image, 2 resting state scans, and 2 diffusion tensor imaging (DTI) scans. Following the imaging protocol, the patients are required to complete another battery of questionnaires, a subset of which are repeated from the Visit 1 to track longitudinal changes in pain. For example, the patients are asked whether they have experienced any changes in health status since the last visit. Additionally, they are asked to verbally recall their average pain levels over the previous 2 weeks, and over the preceding week. This self-reported recalled pain is referred to as “pain memory” and is used as an alternative outcome measure of pain levels.

The method typically further comprises randomization of participants. Optionally, the randomization is performed using 2 kinds of blocks, each with 8 patients; the first block assigns 5 patients to placebo and 3 to no treatment, and the second block assigned 5 patients to placebo, 2 to no treatment, and 1 to active treatment. Each patient ID is randomly attached to a randomization code. Optionally, the initial randomization includes codes for the first few patients, for example, such as 80 patients. It is followed by a second randomization of 50 additional codes about 6 months later. For those assigned to either of the treatment groups, the allocation is performed in a double-blinded fashion. Optionally, a biostatistician performs the randomization; drugs are ordered and re-encapsulated by a regulatory-approved pharmacy, such as the Northwestern Research Pharmacy, and bottled by one or more designated lab members; one or more members of a designated institute (such as the Northwestern University Clinical and Translational Sciences (NUCATS) institute) matches an appropriate treatment drug with patients' randomization code; and study coordinators pick up the blinded agent from NUCATS for storage and dispensing. Furthermore, all drugs are stored at room temperature in a locked cabinet within the lab. The double blind for treatment groups is maintained by the identical encapsulation of the study agent. In an example, a blue pill can be either Naproxen (500 mg) or placebo (lactose) and a bi-colored pill can be either Esomeprazole (20 mg) or placebo. Each person assigned to treatment receives a mixture of blue and bi-colored pills. This way, neither the participants nor the researchers are aware which treatment the participant has received. For those assigned to the no-treatment group, no blind is maintained, as both study staff and participants know that they are not receiving the study agent. Once approximately 50% of all participants have been entered into the study, a preliminary analysis of the electronic pain rating data is completed in order to confirm that there are participants who are experiencing a diminution in pain.

In an embodiment, at the end of Visit 2, participants are randomized into one of three groups: no-treatment, placebo treatment (lactose) or active treatment (the standard of care, which comprises a combination of Naproxen, 500 mg bid, and Esomeprazole, 20 mg bid). A first treatment requires participants in the treatment groups to take a blue pill with a bi-colored pill in the morning and again at night with plenty of water, and record this in their electronic rating app. It will be appreciated that the study staff never informs participants about the odds for receiving active versus placebo treatment—this is important, as the goal is to have participant's own baseline expectations influence whether or not they respond to the placebo treatment. Furthermore, both treatment and no treatment groups continue to receive rescue medication to use if needed, and all participants are required to continue rating their pain twice a day until Visit 3. Optionally, the duration of the first treatment period is approximately 2 weeks long.

Optionally, prospective questionnaire and neuroimaging data uncovers the multiplicity of parameters underlying placebo analgesia. More optionally, machine learning models are used on the prospective questionnaire data and neuroimaging data to classify patients into Resp and NonR and predict their magnitude of response. Notably, the chronic pain patients exhibit identifiable neurobiological and psychological machinery for placebo response in the RCT setting. Optionally, four neuroimaging sessions (Visit 2 to Visit 5) and a proper no-treatment arm is included in the study, which enables distinguishing placebo-related analgesia from non-specific effects.

In an embodiment, on Visit 3, patients are queried about their pain memory, any changes in health since the last visit, and rescue medication usage, any side effects experienced. Beneficially, such information helps the study staff to calculate a treatment compliance of each patient. Participants undergo another 35-minute brain imaging session as performed at Visit 2 and required to complete another set of questionnaires with some repeated from the previous visit. At the end of Visit 3, individuals assigned to the treatment group are told that the study agent would be temporarily discontinued until their next visit so that the effects of the agent could “wash out” of their system. Again, all participants are given rescue medication to use if needed and are asked to continue using their app twice a day until the next visit. Optionally, first washout period is approximately 1 week long.

In an embodiment, at Visit 4, all measurements and procedures from Visit 2 are repeated identically, including the scanning session and questionnaires, and again, queried about their pain memory, rescue medication usage, and changes in health. The study agent is reintroduced to those individuals allocated to one of the treatment groups Optionally, patients are informed that they are receiving the same treatment than the one administered during the first treatment period. All participants are given rescue medication and asked to rate their pain and mood twice a day, as with previous visits. More optionally, the second treatment period is also approximately 2 weeks in length.

In an embodiment, at Visit 5, all measurements and procedures from Visit 3 are repeated identically, including the scanning procedures as on visits 2-4, and filling out a series of questionnaires about their pain, some of which are repeated from the last visits. The participants allocated to a study agent have their treatment discontinued for a second washout period (approximately 1-week long). The participants are asked to use their electronic app twice daily and are given rescue medication if needed.

On average, responders (PTxResp and NoTxResp) showed a diminution in back pain intensity that stabilized to a constant value of about 20% analgesia for both treatment (T1 and T2) and washout periods (W1 and W2). The magnitude of response (% analgesia) showed similar pattern for each treatment period. Importantly, phone app pain ratings at the start of treatment (2 ratings on day 1 of T1) already differentiated placebo treatment responders from the other groups, indicating that observed analgesia is temporally coupled to placebo ingestion only in PTxResp.

In an embodiment, at Visit 6, the last visit, the participants are again queried about their pain memory, changes to health, and rescue medication usage. During this visit, the patients are required to complete a semi-structured, open-ended exit interview with a designated staff member. They are asked more detailed questions about their pain and medical history, quality of life, overall mood, and time in the study. Participants finish with a final battery of questionnaires and return study smartphones, if applicable. There are no scanning procedures on Visit 6. Any ratings submitted for the duration of the study are totaled, and in addition to their visit compensation, participants receive their compensation for the electronic app at the end of Visit 6.

Furthermore, smartphone technology permits tracking fluctuation in pain levels throughout the study. It will be appreciated that two different components of the placebo response: the response or absence of response as well as the magnitude of the response may be required to make the best use of the daily rating data. Optionally, the participants are classified as either Resp or NonR to account for the within-subject variability of pain levels. More optionally, each patient is classified based on a permutation test between the pain ratings acquired during his baseline rating period (Visit 1 to Visit 2) and the pain ratings acquired during his treatment periods (either baseline versus treatment 1, or baseline versus treatment 2). Additionally, the null hypothesis is generated by randomly resampling 10,000 times the distribution of pain ratings, which provides a large set of possible t-values obtained from the rearrangement of the pain ratings. The overall t-value obtained between baseline and treatment is used to determine if the null hypothesis could be rejected (p<0.05) for each of the treatment periods. In the cases where the null hypothesis could not be rejected for either of the treatment periods, the patient is stratified as a NonR. Alternatively, the patient would be stratified as a Responder if there is a significant diminution in the pain ratings. Beneficially, the permutation test takes into consideration the variability across pain ratings during the baseline and treatment periods and represents a statistically-defined cut-off point for response (unlike cut-off points arbitrarily defined by a percentage change in pain).

In an embodiment, a magnitude of response is determined to eliminate the effect of group stratification on the individual responses to placebo treatment. Furthermore, the magnitude of response may be determined by subtracting the averaged pain ratings entered during the baseline period from the averaged pain ratings entered during the last week of each treatment period separately.

Optionally, other outcome measures of pain level, which have been widely used in both randomized clinical trials and research labs, although their utilization in placebo-only trials remains minimal, can be employed. More optionally, measure of pain can be collected using a numerical rating scale (NRS), a memory of pain, a McGill pain questionnaire (MPQ), a neuropathic pain scale (NPS), an affective scale, a sensory scale, pain detect and so forth, and compared with the pain measured using the phone app, to dissociate measurements of intensity from qualities of pain. The NRS represents the traditional standard pain measurement usually used in clinical trials assessing pain levels of participants for both placebo-controlled trials (compared against an active medication) and placebo-only trials (where the placebo effect is being manipulated). The memory of pain represents one of the standard pain assessments used by physicians in clinical practice and correlates well with daily pain diaries in previous studies.

In an embodiment, a phone app pain stratification provides data corresponding to the placebo responders which show consistent 20-30% analgesia in two additional measures of back pain intensity: a numerical verbal recall of their average pain experienced over the last week (pain memory), and a numeric rating scale at time of visit (NRS, commonly used to quantify pain in clinical trials). Qualitative properties of back pain may also be tracked at each visit by one or more questionnaires, such as McGill Pain Questionnaire (MPQ) sensory and affective scales and PainDetect. Such measures do not differentiate between treatment cohorts, and groupings defined by the pain app permutation test shows an overall time effect like that derived from MPQ-sensory score. Therefore, the RCT placebo response is composed of two components: 1) a pill ingestion-related response specifically impacting perceived intensity of chronic pain and 2) a non-specific response reflecting the effect of time or the mere exposure to health care (visits) that modulates qualitative pain measures. Furthermore, mechanisms of the placebo-induced decrease in back pain intensity, using the phone app data, can be determined.

The method typically further comprises employing cell scrambling to further blind the data and minimize bias. All brain imaging and questionnaires data may be analyzed blindly. Cell scrambling may be employed to generate two random labeling of patients, and all group comparisons may be performed three times (e.g., two times for scramble codes and one time for real labeling). Optionally, a lab member not involved in analyses is selected to organize data files and spreadsheets for processing and statistical analyses of the data. More optionally, the lab member first renames all the data files in order to ensure that analysts are blinded to each participant's unique ID, and to minimize bias from previous interactions with patients during data collection. Next, all analyses are performed with 3 randomized codes (namely, “classifiers”) for each condition, with only one of them being the proper classification of placebo treatment responders, non-responders, and no treatment responders and non-responders. Furthermore, the selected lab member performs a “triple blinding” (because analyzers are blind to participant ID, participant treatment, and correct participant group classification) prior to any analyses, with the exception of the pain ratings from the app, which are used to stratify patients from the outset. As a result, each analysis is done three different times in an unbiased manner. Importantly, the three lab members who contributed to the analyses are not informed that they are provided different classifiers to make sure they do not collaborate to figure out which one is the real code. The results are presented in a public lab meeting where the lab member un-blinds the analyzers to the data to confirm which results are true. Specifically, the outcomes and data from the correctly classified group (out of the 3 classifiers) in each instance are presented. Although, results from the 2 false classifiers are presented where applicable in for the purpose of comparison. After completing all the analyses, the real labeling of patients is properly identified based on the significance of results. Beneficially, this procedure aims to decrease uncontrolled bias during data analyses and to enhance the reproducibility of results.

Optionally, the brain imaging procedure is completed in about 35 minutes, however, an extra 25 minutes may be allocated to install the patients in a comfortable position to keep their back pain at a minimum, and to re-acquire images if the data is contaminated by head motion. More optionally, a high-resolution T1-weighted brain images are collected using integrated parallel imaging techniques (PAT; GRAPPA) representing receiver coil-based data acceleration methods. The acquisition parameters are: isometric voxel size=1×1×1 mm, TR=2300 ms, TE=2.40 ms, flip angle=9°, acceleration factor of 2, base resolution 256, slices=176, and field of view (FoV)=256 mm. The encoding directions are from anterior to posterior, and the time of acquisition is 5 min 21 sec.

Optionally, blood oxygen level-dependent (BOLD) contrast-sensitive T2-weighted multiband accelerated echo-planar-images are acquired for resting-state fMRI scans. Multiband slice acceleration imaging acquires multiple slices simultaneously, which permits denser temporal sampling of fluctuations and improves the detection sensitivity to signal fluctuation. The acquisition parameters are: TR=555 ms, TE=22.00 ms, flip angle=47°, base resolution=104, 64 slices with a multiband acceleration factor of 8 (8×8 simultaneously-acquired slices) with interleaved ordering. High spatial resolution is obtained using isomorphic voxels of 2×2×2 mm, and signal-to-noise ratio is optimized by setting the field of view (FoV) to 208 mm. Phase encoding direction is from posterior to anterior. The time of acquisition lasts for about 10 min 24 sec, during which 1110 volumes are collected. Patients are instructed to keep their eyes open and to remain as still as possible during acquisition. More optionally, the procedure is repeated two times.

Optionally, a pre-processing of functional images is performed. More optionally, the pre-processing is performed using FMRIB Software Library (FSL) and an in-house software. In such scenario, the first 120 volumes of each functional dataset are removed in order to allow for magnetic field stabilization. This leaves a total of 990 volumes for functional connectivity analyses. The effect of intermediate to large motion is initially removed using fsl_motion_outliers. Time series of BOLD signal are filtered with a Butterworth band-pass filter (0.008 Hz<f<0.1 Hz) and a non-linear spatial filter (using SUSAN tool from FSL; FWHM=5 mm). Following this, six parameters, obtained by rigid body correction of head motion, global signal averaged over all voxels of the brain, white matter signal averaged over all voxels of eroded white matter region and ventricular signal averaged over all voxels of eroded ventricle region, are regressed. These nine vectors are filtered with the Butterworth band-pass filter before being regressed from the time series. Finally, noise reduction is completed with Multivariate Exploratory Linear Optimized Decomposition into Independent Components (MELODIC tool in FSL) that identified components in the time series that are most likely not representing neuronal activity. Beneficially, such conservative ICA regression process removes only components related to motion or noise.

Optionally, the functional image is registered. The functional image registration is optimized according to a two-step procedure, wherein the two-step procedure comprises: 1) all volumes of the functional images to be averaged within each patient to generate a contrast image representative of the 990 volumes and 2) linearly registering such functional image to the MNI template and averaging a common template across patients to generate a common template specific to our CBP patients. Finally, all pre-processed functional images are non-linearly registered to the common template using FNIRT tool from FSL. Additionally, the registered brains are visually inspected to ensure optimal registration.

Optionally, a parcellation scheme is performed, wherein the brain can be divided into 264 spherical ROIs (5-mm radius) located at coordinates showing reliable activity across a set of tasks and from the center of gravity of cortical patches constructed from resting state functional connectivity. Because limbic structures are believed to play a role in placebo response, 5-mm radius ROIs are manually added in bilateral amygdala, anterior hippocampus, posterior hippocampus and NAc. Linear Pearson correlations are performed on time courses extracted and averaged within each brain parcel. Given a collection of 272 parcels, time courses are extracted to calculate a 272×272 correlation matrix. These matrices allow for the construction of weighted brain networks, where nodes represent brain regions and links represent weighted connections from Pearson correlations between any given set of these regions.

Optionally, consistent community structures across a large number of network partitions is determined, using Louvain algorithm integrated in the Brain Connectivity Toolbox (BCT; https://sites.google.com/a/brain-connectivity-toolbox.net/bct/). For each subject, the individual community structure is initially constructed from 100 repetitions of the same network. The group community is then constructed from 100×63 patients, generating a total of 6300 networks. The final community structure is created by thresholding the averaged within-module connectivity likelihood matrix at 0.5, meaning that if the likelihood for two nodes belonging to the same module is above 50%, they are considered in the same module. This allows identifying six separate communities, including the four communities of interest.

Optionally, localizers from an independent data set consisting of osteoarthritis patients where placebo response is predicted from resting state fMRI functional connectivity is used to identify communities of interest. Furthermore, resting state functional connectivity is used to identify four regions predicting patients in the placebo arm that responded to treatment: the right mid-frontal gyms connectivity (x=28, y=52, z=9), the anterior cingulate cortex (x=−3, y=40, z=2), the posterior cingulate cortex (x=−1, y=−45, z=15), and the right somatosensory cortex (x=60, y=−7, z=21). The coordinates are used as seeds in an analytic tool, such as Neurosynth analytic tool (http://neurosynth.org), and three networks sharing strong connectivity with these seeds: the DMN, the frontoparietal network, and the sensorimotor network are extracted. Communities corresponding to these networks are identified based on spatial overlap, by multiplying the networks of interest with the nodes pertaining to each community. In an example, a total of 113 nodes may be affiliated with these communities. The 151 nodes affiliated with the visual and saliency communities and those nodes without affiliation to any community may be excluded from the analyses. The limbic nodes and a node located in the PAG from the Power parcellation scheme (which is not affiliated with any community) may be added for a total of 122 nodes of interest. This approach is part of our initial analysis design because it has many advantages, including increasing statistical power by limiting the number of comparisons, preventing over-fitting of the data, and identifying hypothesis-driven functional networks with the potential of generalizing obtained results across different chronic pain conditions.

Optionally, network statistics are performed to identify brain networks predisposing to placebo response (performed on the 122×122 connectivity matrix). Group differences are examined using a permutation test (5,000 permutations) on the connections of the weighted network (122*121 nodes), controlling for false discovery rate (FDR p<0.05) using the Network Based Statistics toolbox (NBS). The fisher-z transformed correlation coefficients z(r) of the significant connections are extracted at each visit and entered in a repeated measured ANOVA testing for an interaction of time with placebo treatment response.

Optionally, a grey matter density is examined using voxel-based morphometry from FSLVBM. All T1-weighted images are first brain extracted and then segmented into grey matter, white matter, or cerebrospinal fluid. A common grey matter template is generated for CBP by registering and averaging all grey matter images. The grey matter image of each participant is then registered to the common template using non-linear transformation. A voxel-wise permutation test is used to test the significance of group differences between placebo responders and non-responders to a distribution generated from 5000 permutations of the data for each voxel of the template, using a sigma filter of 3 mm for smoothing. The initial analysis established significance level using the Threshold-Free Cluster Enhancement (TFCE) method (FWE p<0.05).

Optionally, cortical thickness is examined using Freesurfer software library (http://surfer.nmr.mgh.harvard.edu/). The structural processing includes skull stripping, intensity normalization, Taliarch registration, segmentation of the subcortex, reconstruction of the cortical surface, and tessellation of the gray/white matter boundary and pial surface. Following reconstruction of the cortical surface, brains are inflated, averaged across participants to produce a study-specific brain, and then smoothed using a 10 mm full-width at half maximum Gaussian kernel. A direct measure of cortical thickness is calculated using the shortest distance (mm) between the pial surface and gray-white matter boundary at each point or vertex. Cortical thickness analysis for each hemisphere is conducted using FreeSurfer's Query, Design, Estimate, Contrast (QDEC) graphical interface. The initial vertex-wise comparison is performed between placebo responders and non-responders for each hemisphere. Correction for multiple comparisons is performed using random-field-theory-based significant clusters at p<0.05. Values of cortical thickness are extracted in the significant cluster surviving multiple comparison and compared between PTxNonR, PTxResp, NoTxNonR, NoTxResp groups using a one-way ANOVA. The values of cortical thickness in the significant cluster are extracted at each visit and entered in a repeated measured ANOVA.

Optionally, volumetric analyses of T1-weighted images are performed through automated processes using both FSL (version 5.0.8) and FreeSurfer (version 6) software. The volume differences in 3 subcortical nuclei selected a priori; the NAc, the amygdala, and the hippocampus is investigated. After using FSL's brain extraction tool (BET) to remove the skull from all images, FSL's integrated registration and segmentation tool (FIRST) is utilized to segment these specific subcortical regions and extract their volume measurements. Unilateral volume measurements for each region are initially compared between responders and non-responders. Given the recent evidence from the ENIGMA consortium showing that subcortical volume asymmetry can provide a brain signature for psychopathologies, a possibility that asymmetry differences may provide a biomarker for placebo propensity is also investigated in the data. All subcortical regions' volumes are summed for the right and the left hemisphere separately; for each patient, the ratio between the two (right/left) is created, where a result=1 indicates a perfect subcortical symmetry, whereas numbers>1 or <1 indicates an asymmetry biased toward the right or left hemispheres, respectively. More optionally, volumes and subcortical asymmetry are compared between PTxNonR, PTxResp, NoTxNonR, NoTxResp using a one-way ANCOVA controlling for peripheral grey matter volume, age and sex. The effect is tested across all visits using repeated measure ANCOVA controlling for peripheral grey matter volume, age, and sex.

Optionally, a permutation test is performed on the weighted 7,381 resting state connections between all possible pairs of nodes within the modules of interest (FDR-corrected p<0.05). The edges differentiating the two PTx response groups are all connected to nodes located in the DLPFC. Precisely, PTxResp displayed stronger connections for the link DLPFC-preCG, and weaker connections for links DLPFC-rACC and DLPFC-PAG. As expected, all three networks differentiated PTxResp from PTxNonR at visit 2. Moreover, the DLPFC-preCG and the DLPFC-rACC connectivity showed neither a main effect of time nor an interaction effect of group*time, indicating that these connections represent time-invariant mechanisms that differentiate PTxResp from PTxNonR across all visits. On the other hand, the initial differences between groups of MFG-PAG connections dissipated by visit 4. These results therefore demonstrate the existence of a DLPFC-functional network, whose components either stably or transiently determine the likelihood of placebo response. Importantly, each component of this DLPFC-functional network also tracked the magnitude of placebo response. Performing this analysis using scramble codes for labeling patients generated no significant group differences.

In an embodiment, the volumes of the NAc, amygdala, and hippocampus are first examined because they represent risk factors for developing pathological emotional states and chronic pain. Comparing subcortical volumes between PTxResp and PTxNonR is not informative. Inter-hemispheric laterality of the combined volume of these three structures, however, indicated that PTxResp showed rightward limbic volume asymmetry compared to PTxNonR, and this asymmetry is observed in all four visits/scans. Importantly, this result is validated using another brain segmentation software, such as Freesurfer and the like. The differences in anatomical properties of the cortex are assessed with grey matter density and cortical thickness. Whole-brain cortical thickness measurements show that PTxNonR has thicker cortex in the right superior frontal gyrus than PTxResp. The identification of brain morphological features, present before treatment and persisting throughout the study, provides evidence for placebo propensity stemming, in part, from stable brain biology. Here again, the scramble codes yielded no significant group differences.

Furthermore, in an embodiment, psychological parameters predisposing CBP patients to the placebo response are identified from a battery of 15 questionnaires with 38 subscales collected at visit 1. A covariance across all factors used to assess personality and psychological states is identified. Univariate statistics is used to assess group differences and correlations with the magnitude of response. While none of the group contrasts would survive correction for multiple comparisons, the results indicated that a number of subscales from the Multidimensional Assessment of Interoceptive Awareness (MAIA) questionnaire are tightly coupled with the magnitude of response, as is the quality of “openness” from the Neo-5 personality dimensions. In particular, Emotional Awareness (MAIA/e) and Not Distracting (MAIA/nd) are strongly correlated with % analgesia.

The method typically comprises analysis of the questionnaire data. Optionally, over the course of the 6 visits (Visit 1 to 6), 29 unique questionnaires filled out by the participants are analysed. More optionally, these specific self-report measures are chosen for one of 4 reasons: (1) to gather basic information about participants, including demographics and pain/medical history, (2) to track any changes in the quality and/or intensity of pain characteristics as measures of treatment efficacy, (3) to monitor any changes in emotional affect which may have influenced someone's time in the study or their treatment response, and (4) to capture trait-based qualities, general habits and beliefs, or state-related expectations of individuals that may predispose them to respond to placebo. Questionnaires associated with tracking pain and mood changes overtime are repeated across all study visits. Questionnaires associated with expectations towards treatment and satisfaction after treatment are conducted twice—either before treatment sessions (visits 2 and 4) or after treatment periods (visits 3 and 5), respectively. In contrast, measures corresponding to identifying more stable traits of participants are completed at visit 1, which allows a possible prediction of response. Finally, a subset of questionnaires regarding beliefs toward alternative medicines and suggestibility are administered at the final visit after the exit interview. Furthermore, predictors of placebo response can be analysed using the data from the questionnaires (except the pain questionnaires collected at every visit to determine treatment outcome) collected at visit 1 only.

Furthermore, data from these self-report measures may be downloaded directly from REDCap as a CSV file and scored in Excel according to their references. Optionally, the questionnaires comprise an option to “skip” a question, in case the participant does not feel comfortable answering a certain item. If more than 20% of the data from a given questionnaire is missing, the person's data for the questionnaire is not scored; for all other missing data, the mean is used to fill in missing items as used in data analysis to conserve statistical power for a relatively small sample size. It will be interesting to note that of all the self-report data analyzed, less than 3% is totally missing and thus unable to be filled in as described above.

In an embodiment, the magnitude of response is predicted using just the combination of questionnaire data. The scales contributing to the prediction of response magnitude are: 1) the Emotional Awareness and Not-Worry subscales of the MAIA Questionnaire (MAIA/e; MAIA/nw); 2) the Non-painful Situations subscale of the Pain Sensitivity Questionnaire (PSQ/np); 3) the Describing subscale of the Five Facets of Mindfulness Questionnaire (FFM/d); and 4) Openness from the NEO-FFI. The model is no longer able to predict the magnitude of response after removing these psychological parameters. This not only indicates their importance for response, but also reveals that neither the traditional personality measures reported in healthy controls under placebo conditioning (e.g., neuroticism, extraversion, optimism) nor any of the chronic pain related personality traits usually linked to severity of symptoms (e.g., anxiety, catastrophizing, and fear of pain) contributes to the prediction.

Optionally, machine learning is employed to test the predictive value of brain imaging and questionnaires data. Moreover, machine learning is used to determine if placebo response could be predicted from prospective brain imaging and questionnaires data. More optionally, a nested leave one out cross validation (LOOCV) procedure is implemented, wherein placebo outcome of each patient is predicted using an independent training sample. Specifically, SVM models are trained in an inner loop (n=42) and applied to a left-out participant. Within each n−1 patients training sample set, the model parameters are tuned using 10-fold cross-validation. Within each training sample set, the scores of the normalized 38 subscales are used to build the SVM classifier. SVM classification has an accuracy of 0.72 in classifying placebo response [95% CI, 0.56 to 0.85]. Sensitivity of this approach is 0.73 (95% CI, 0.52 to 0.88), and specificity is 0.71 (95% CI, 0.44 to 0.90). This level of accuracy is higher than chance level determined by a null distribution from 100 scramble labels (0.72 accuracy corresponded to a z-score of 2.68). Furthermore, the purpose of the inner loop is to optimize the parameters of the model through cross validation. Once the optimal model showing the least amount of error is identified, it is applied to the left-out participant to either classify the patient as a PTxResp or PTxNonR or predict the magnitude of his response. Beneficially, such procedure can be applied to build predictive model from rsfMRI, brain anatomy and personality independently.

Optionally, a classifier is trained using brain imaging data entering either brain anatomy or rsfMRI as predictors. However, such classifier failed at classifying PTxResp and PTxNonR above chance level (with accuracies <than 0.67, which corresponds to a z-score of 1.96). Moreover, adding features from rsfMRI or brain anatomy to the questionnaire data does not improve the classifier's accuracy (accuracy of 0.65). Therefore, feature selections and data reduction procedures used in the training sample set are required to improve the classifier's accuracy.

Optionally, feature selection is achieved by the rsfMRI model. The rsfMRI model is built based on the weighted 7,381 connections from the matrices used for network analyses. More optionally, feature selection is initially performed within the inner loop using several data reduction strategies: principal component analyses (PCA; 42 components), unsupervised machine learning (Corex; 40 variables), or averaged connectivity within and between communities. Additionally, features selection is also performed using univariate t-test on each connection to identify group differences (PTxResp Vs PTxNonR within the raining set; p<0.001) or links correlating with the magnitude of response (robust regression with % anagesia within the raining set; p<0.001).

Optionally, three different measures are used to predict the magnitude of response using brain anatomy: 1) the averaged cortical thickness in the 74 labels per hemispheres from the Destrieux Atlas, 2) volumes of the 16 subcortical structures segmented with FSL FIRST, 3) subcortical volume asymmetry (ratio Right/Left) of these 16 subcortical structures. Optionally, the data is normalised as the features are derived from different measurements. Additionally, the data is also normalised as all the questionnaires are on different scales. Furthermore, all the 38 items of questionnaire collected at Visit 1 are entered in the rsfMRI model.

In an embodiment, brain networks constructed from rsfMRI are examined to directly identify functional connectivity that predisposes patients' response prior to placebo treatment. Based on previous findings, placebo-related networks of interest from results in OA patients exposed to placebo treatment in an RCT are derived. Furthermore, a modularity analysis segregating the functional networks into 6 communities is also performed, and all analyses are restricted to the default mode network (DMN), sensorimotor (SM), and frontoparietal (FP) communities, due to their overlap with placebo-related networks observed in OA. Subcortical limbic regions can be added along with the PAG because of their involvement for placebo response and pain chronification. No other exploratory analyses is required to be performed.

Furthermore and optionally, it may be determined that if rsfMRI is collected prior to placebo ingestion, it could predict the magnitude of response. Within each n−1 patients training sample set, a feature selection is performed to identify links correlating with the magnitude of response (robust regression, p<0.001) prior to the LASSO regression. These connections are used to train a predictive model using 10-fold cross-validation for tuning the LASSO parameters. The model is then tested in the left-out patient. Because the number of features and weights differ between each loop, a “consensus” is generated by averaging the weights across the n=43 loops to create a final single set of weights. The resultant network consists of a combination of 19 weighted connections predicting the magnitude of placebo analgesia. These include links connecting nodes located in bilateral amygdala, the posterior hippocampus, the PAG, the temporo-parietal junction (TPJ), the DLPFC, and the orbitofrontal cortex (OFC). Optionally, linear regression entering the predicted magnitude of response from rsfMRI and the one from questionnaires data reveals that both models explain independent variance of the actual response, suggesting that they are complementary one to another.

Although this set of data may be predictive of magnitude of response prior to the first placebo treatment, the prediction may not generalize to rsfMRI data collected at other visits post treatment. This may be due to the small number of connections in the present study, which are likely to be changing in time as a consequence of learning and adjustment of expectations, especially after the introduction of a placebo treatment. Because the model may not be stable across the post-treatment visits, its capacity for predicting magnitude of response in a new set of chronic pain patients may remain to be determined in further studies. The procedure is nevertheless informative regarding the potential involvement of additional neurophysiological contributors to the placebo response.

Optionally, Support vector machine (SVM) is used to discriminate between PTxResp and PTxNonR using fitcsvm function implemented in Matlab. More optionally, a nested leave one out cross validation (LOOCV) procedure is implemented, where SVM models are trained in an inner loop (n=42) and applied to a left-out participant. The box constraint and the radial basis function (rbf) kernel are optimized through a 10 folds cross validation strategy within the inner loop. Additionally, once the optimal SVM model is identified, it is applied to classify the left-out patient as a PTxResp or PTxNonR.

Optionally, magnitude of response is predicted using Least Absolute Shrinkage and Selection Operator (LASSO) regressions to train a model for predicting the magnitude of response. More optionally, a nested LOOCV procedure is used, where SVM models are trained in an inner loop (n=42) and applied to a left-out participant. The inner loop determines feature selection and lambda regularization parameters using 10 folds cross-validation. The generalization error is estimated by testing the model to the left-out patient. The procedure is performed using the lasso function implemented in Matlab.

In an embodiment, Least Absolute Shrinkage and Selection Operator (LASSO) regression is used to predict the magnitude of placebo response (continuous variable approach) using a nested LOOCV procedure. The model is trained in n−1 patients in an inner loop using 10 folds cross-validations for tuning the LASSO parameters, and then tested in the unseen held out patient.

In an embodiment, the neuroimaging-based placebo RCT is conducted in 129 CBP patients. However, from the initial 129 chronic back pain (CBP) patients recruited in the study, 4 individuals are assessed for eligibility but met exclusion criteria before consenting. Of the enrolled 125 patients, 43 failed to meet the inclusion criteria at Visit 1 or during the 2-week baseline period between Visits 1 and 2. The remaining 82 patients are randomized into one of three groups: no treatment (n=25); active treatment (n=5); or placebo treatment (n=57). Furthermore, of the no treatment group, n=5 either discontinued from the study or lost to follow up; of the placebo treatment group, n=11 either discontinued or lost to follow-up, with an additional 2 participants being excluded from final analysis due to having average baseline pain rating values below 4/10. Furthermore, the participants are required to visit the lab on six occasions (6 visits, Visit 1 to Visit 6) over 8 weeks and undergo identical scanning (brain imaging/screening) protocols on four out of six visits (Visit 2 to Visit 5). Additionally, throughout the duration of the study, participants use a visual analogue scale, displayed on a smartphone (or computer) app, to rate back pain intensity two times per day in their natural environment. The inclusion of an active treatment group is used to ensure that the double blind for placebo treatment is maintained for the duration of the study. Therefore, the 5 participants randomized in the active treatment group are not analyzed. Furthermore, an analysis is formulated for eligibility based on 63 out of 129 CBP patients. 63 patients are dichotomized into Resp and NonR based on a within-subject permutation test performed on the pain app ratings to determine improvement of symptoms. The final sample size includes 43 CBP patients randomized to the placebo treatment group (patients receiving placebo treatment (PTx)) showed a diminution in pain intensity as compare to 20 CBP patients randomized to the no treatment group (patients from the no treatment arm (NoTx)) controlling for regression to the mean, spontaneous variations in symptoms, and placebo response to other cues than the pills. A cross-validated model derived from rsfMRI predicts the magnitude of the placebo response with accuracy scores equivalent to predictive models developed from functional brain imaging in healthy individuals. Furthermore, the participants may be compensated a predefined sum of money (such as $50) for each visit completed, and reimburse a predefined sum of money (such as up to $20) for travel and parking expenses, if applicable.

The following examples are set forth as being representative of the present disclosure. These examples are not to be construed as limiting the scope of the present disclosure as these and other equivalent embodiments will be apparent in view of the present disclosure, figures and accompanying claims.

### Example I

Testing the Placebo Effects on Patients.

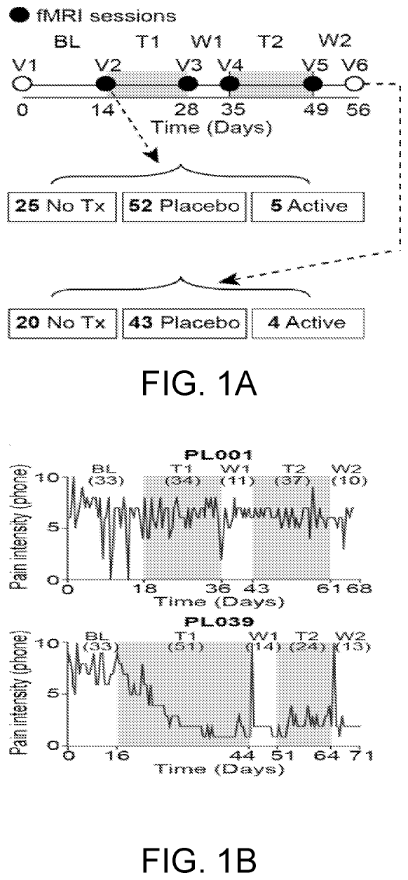

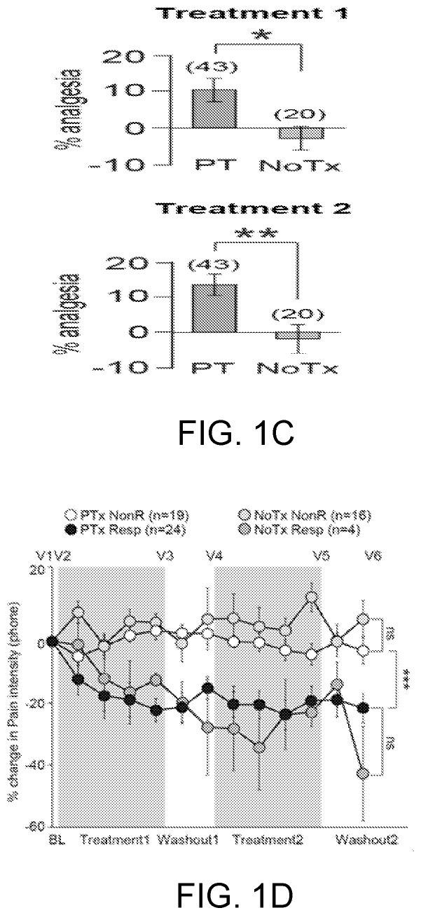

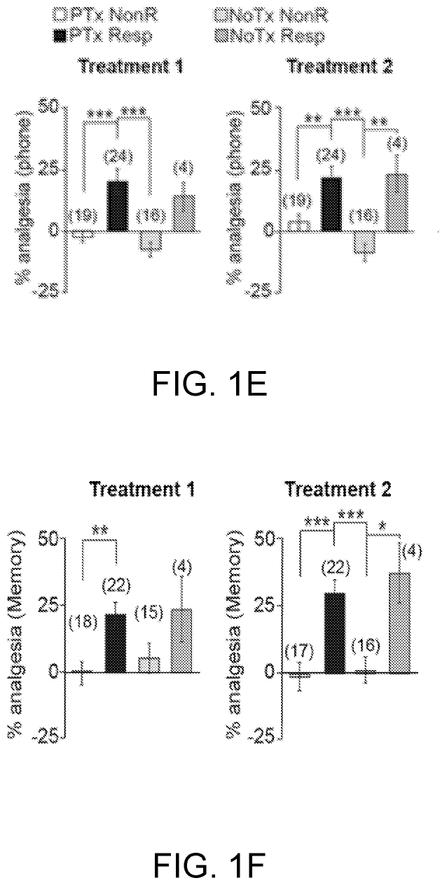

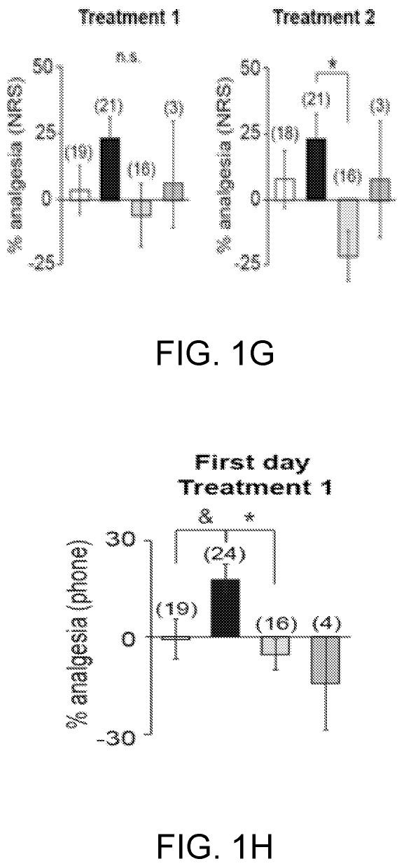

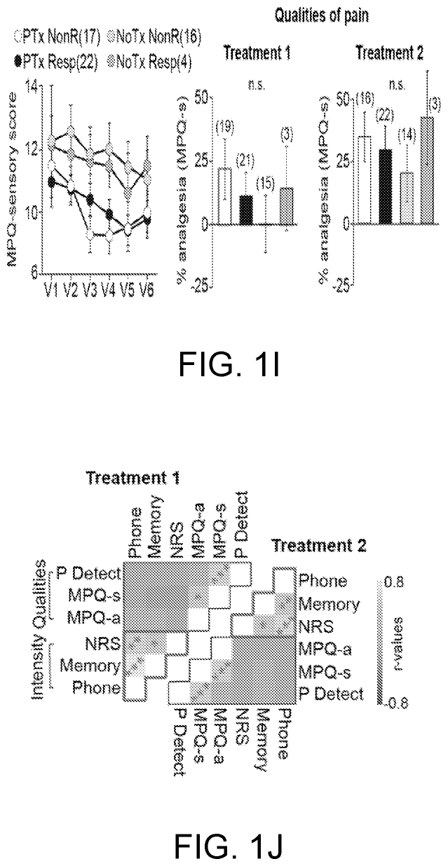

Referring now to the figures, FIGS. 1A-1J illustrate methods for testing the placebo effects on patients in a clinical trial and the results of such methods, in accordance with one embodiment of the present disclosure. The methods and results illustrated in FIGS. 1A-1J demonstrate that the placebo diminishes back pain intensity while trial participation non-specifically decreases qualitative pain outcomes. Referring to FIG. 1A, and the experimental design and time line: CBP patients entered a six-visit (V1-6) 8-week randomized controlled trial, including baseline (BL), treatment (T1, T2), and washout (W1, W2) periods. Participants entering and completing the study are indicated. Referring to FIG. 1B, exemplary time series of smartphone app pain ratings in 2 patients (values in parentheses are numbers of ratings) is shown. Referring to FIG. 1C, the patients receiving placebo treatment (PTx) showed lower pain levels during the last week of each treatment period compared to the patients in the no treatment arm (NoTx) is shown. Referring to FIG. 1D, subject permutation tests between pain ratings entered during BL and T1 or T2 identified responders in PTx and NoTx groups is shown. The group-averaged % change in phone app ratings of back pain intensity (2 bins/week) is displayed in therein. The results showed a group by time interaction, indicating that PTxResp and NoTxResp showed improvement of symptoms, and that placebo treatment increased the frequency of response compared to the no treatment group. Referring to FIGS. 1E, 1F and 1G, shown is that pain intensity decreased by about 20-30% for all intensity tracking outcomes (phone, memory, numerical rating scale (NRS)) during both treatment periods. No-treatment responders (NoTxResp) showed similar magnitude analgesia as the placebo responders, and no-treatment non-responders (NoTxNonR) approximated placebo non-responders. FIG. 1H, illustrates that the placebo analgesia is present the first day after placebo ingestion, only in placebo responders. In FIGS. 1C-1H, the number of subjects are in parentheses. All post hoc comparisons are Bonferroni corrected: & p<0.10; * p<0.05; p<0.01 *** p<0.001, n.s. not significant. Error bars indicate SEM. FIG. 1I, illustrates that the qualitative outcome measures (McGill pain questionnaire, MPQ-sensory) decreased in time similarly in all groups. FIG. 1J, illustrates that the principal component analyses clustered pain outcomes into 2 factors, segregating intensity and quality. Group by time by pain (Intensity Vs Quality) interaction indicated that only the pain intensity is diminished by placebos.

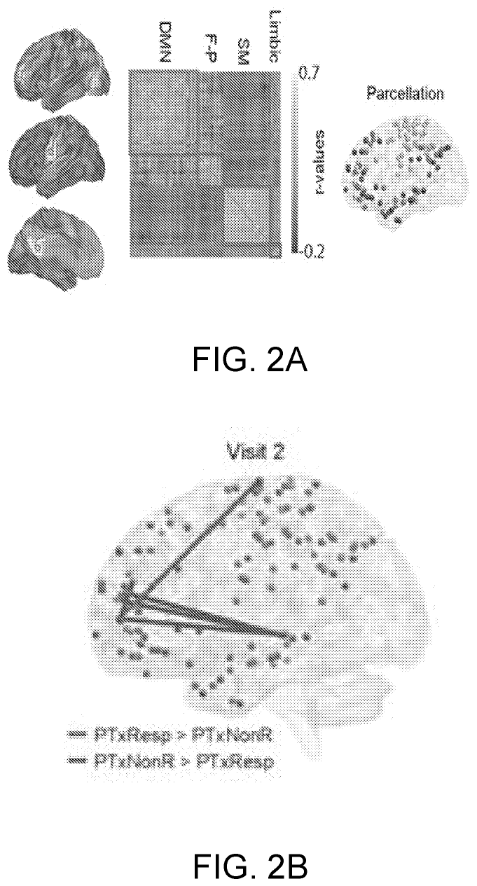

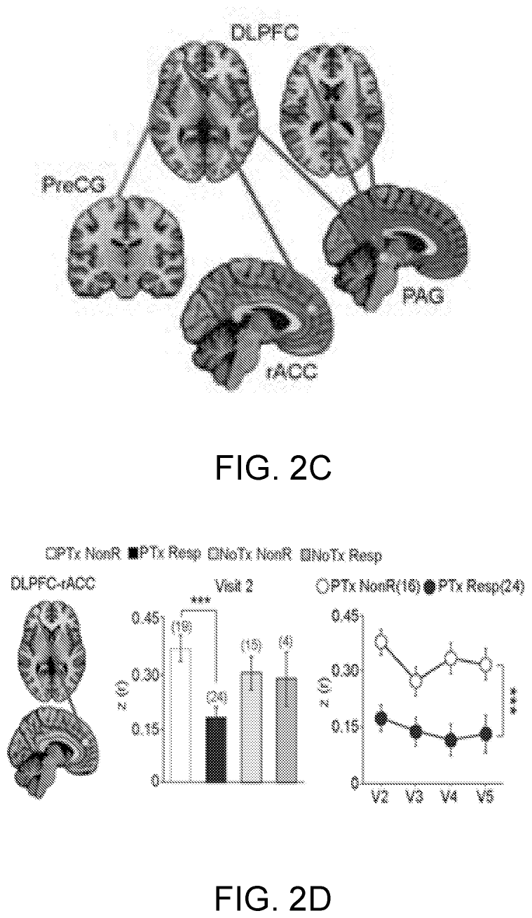

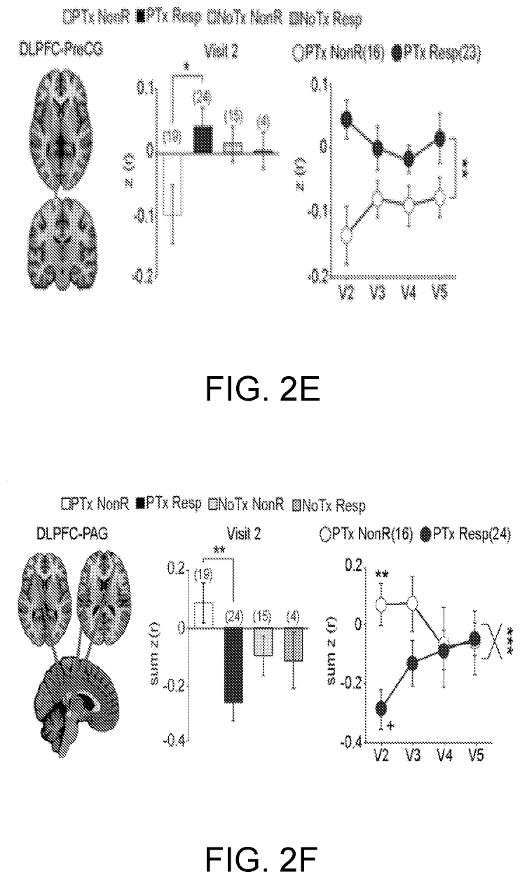

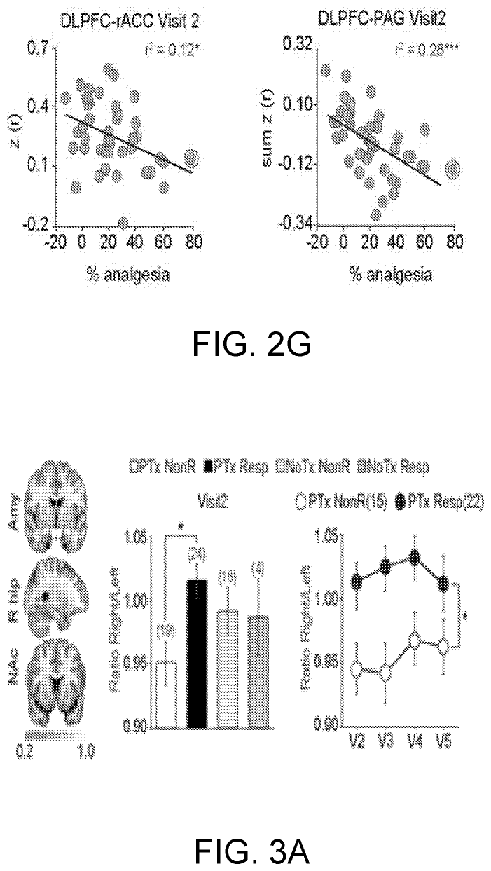

FIGS. 2A-2G illustrate the effects of a placebo on the dorsolateral prefrontal cortex network of patients, in accordance with an embodiment of the present disclosure. In particular, FIGS. 2A-2G describe the placebo response that identifies the dorsolateral prefrontal cortex (DLPFC) functional network with invariant and transient components. Referring to FIG. 2A, previous results indicate that functional connectivity in four regions: middle frontal gyms (MFG), rostral-anterior and posterior cingulate cortex (rACC, PCC), and sensorimotor (SM) cortices predict placebo response in OA patients. Co-activation maps derived from these seeds (green) (1000 healthy subjects; http://neurosynth.org) are used to restrict brain networks of interest. The connectivity matrices of our data are restricted to communities overlapping with these networks of interest: default mode network (DMN), frontoparietal (F-P), sensorimotor (SM), and limbic. FIGS. 2B-2C illustrate that the functional networks at visit 2 distinguishes PTxNonR and PTxResp. Stronger connectivity link Dorsolateral prefrontal cortex (DLPFC)-Precentral gyrus (PreCG) and weaker connectivity links MFG-rACC, and MFG-periaqueductal grey (PAG) identified placebo responders. FIGS. 2D-2F illustrate that MFG-PreCG and MFG-rACC connections differentiated placebo responders and remained constant in time while MFG-PAG connectivity differentiated placebo response transiently (interaction time with group trending p=0.09). FIG. 2G, illustrates that each of these brain parameters correlated with the magnitude of the response. All post hoc comparisons are Bonferroni corrected: * p<0.05; ** p<0.01; *** p<0.001; + within comparison V2 vs. V5: p<0.05. Error bars indicate SEM. The red circle marks an outlier on the magnitude of response and is excluded from the % analgesia correlation analyses.

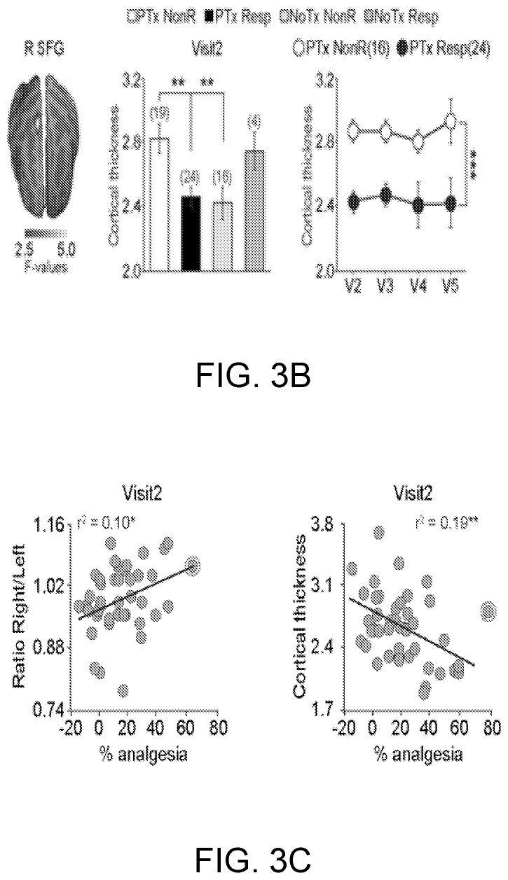

FIGS. 3A-3C illustrate the effects of a placebo on the subcortical limbic volume asymmetry and sensorimotor cortex, in accordance with an embodiment of the present disclosure. FIG. 3A provides heat maps displaying overlap of automated segmentation for nucleus accumbens (NAc), right hippocampus (R hip), and amygdala (Amy) across all patients. The placebo responders displayed a rightward asymmetry in overall volume of subcortical limbic structures after controlling for peripheral grey matter volume, age, and sex at visit 2. The placebo group-dependent asymmetry is observed at all 4 visits/scans. FIG. 3B illustrates that the whole-brain neocortical vertex-wise contrast indicated thicker sensorimotor, right superior frontal gyrus (R SFG), in PTxNonR. This effect is consistent across all 4 visits/scans. Referring to FIG. 3C, the anatomical properties correlated with the magnitude of the response. All post hoc comparisons are Bonferroni corrected: p<0.05; p<0.01; p<0.001. Error bars indicate SEM. The red circle marks an outlier on the magnitude of response and is excluded from the % analgesia correlation analyses.

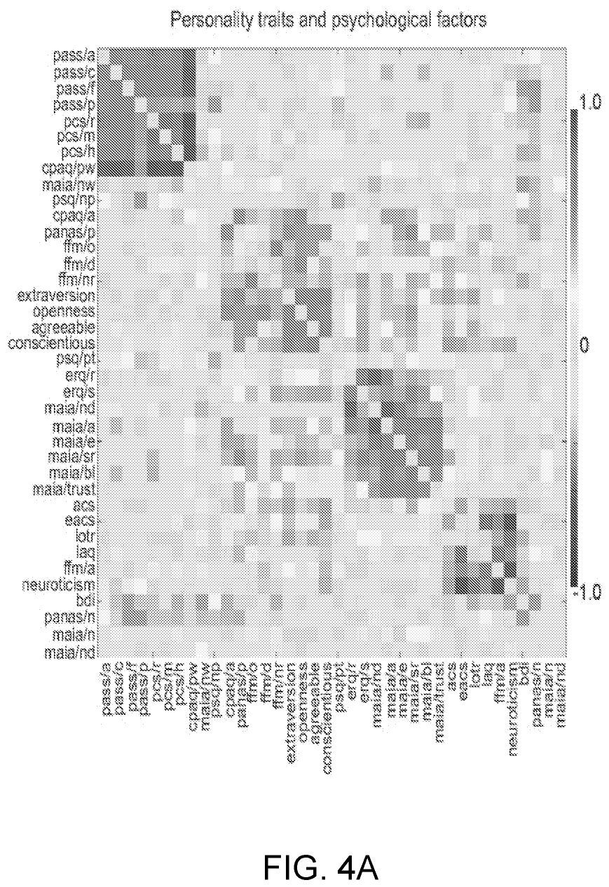

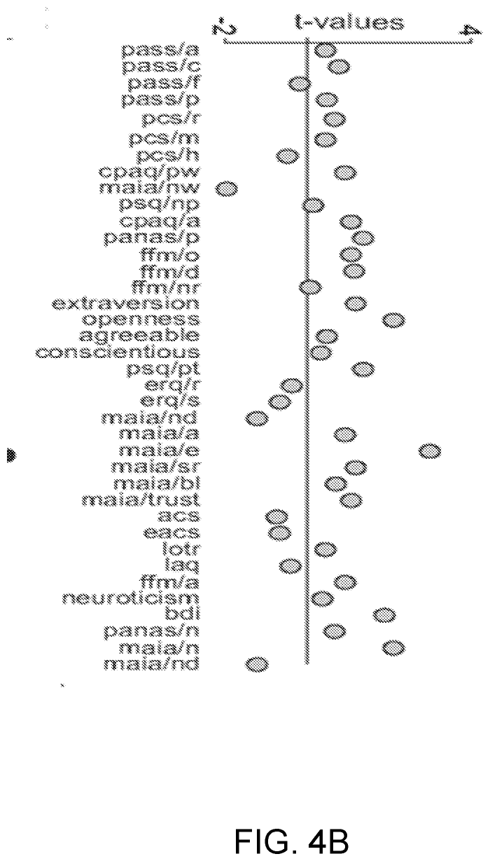

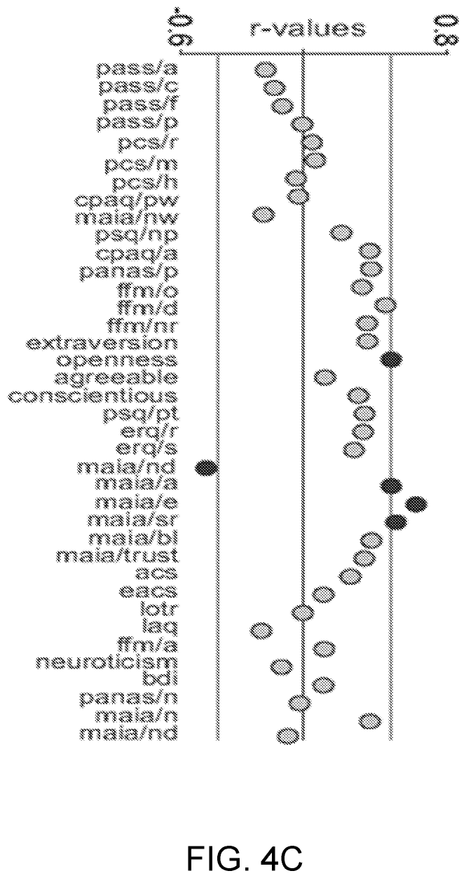

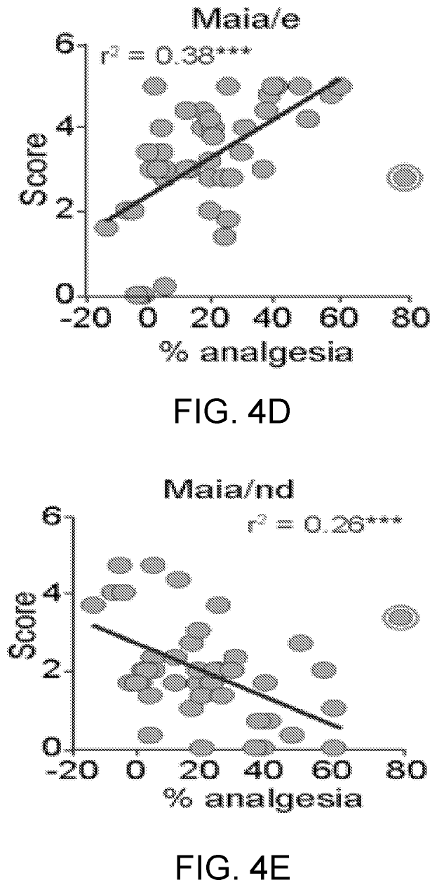

FIGS. 4A-4E illustrate personality traits and psychological factors of a patient in response to a placebo, in accordance with an embodiment of the present disclosure. FIG. 4A, illustrates a covariance matrix that describes the relationship between each subscale in the questionnaire data. Referring to FIG. 4B, illustrates that univariate statistics showed no group differences surviving multiple comparisons. FIG. 4C illustrates that the openness (from the big 5 personality dimensions) and 4 subscales from the Multidimensional Assessment of Interoceptive Awareness (MAIA)—Not Distract, Attention Regulation, Emotional Awareness, and Self-Regulation—correlated with the magnitude of response after correcting for multiple comparisons (p<0.0013; shown in black). Maia/e, Mais/nd are displayed therein. FIGS. 4D-4E illustrate that the red circle marks an outlier on the magnitude of response that is excluded.

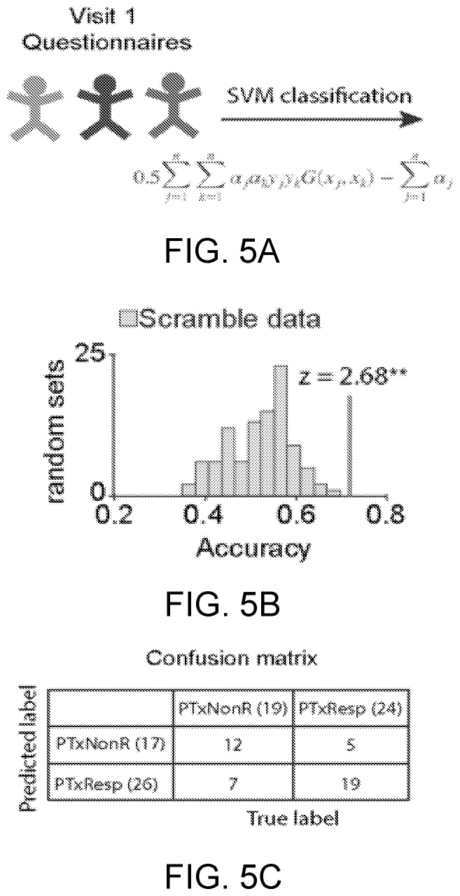

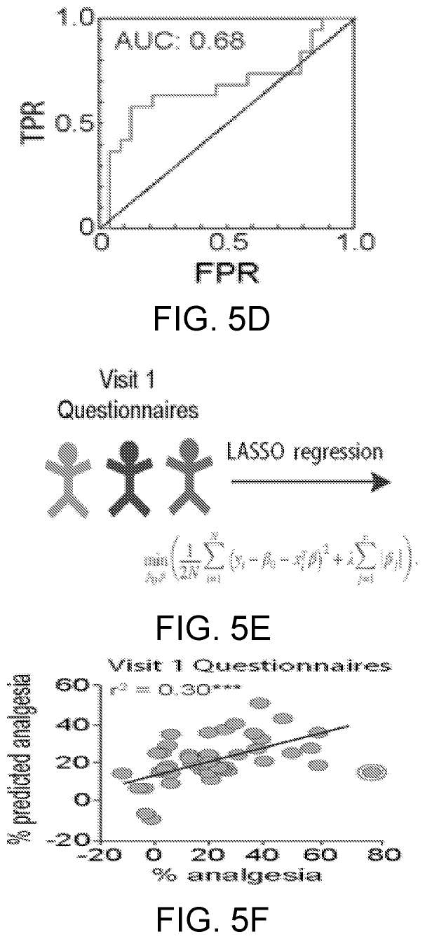

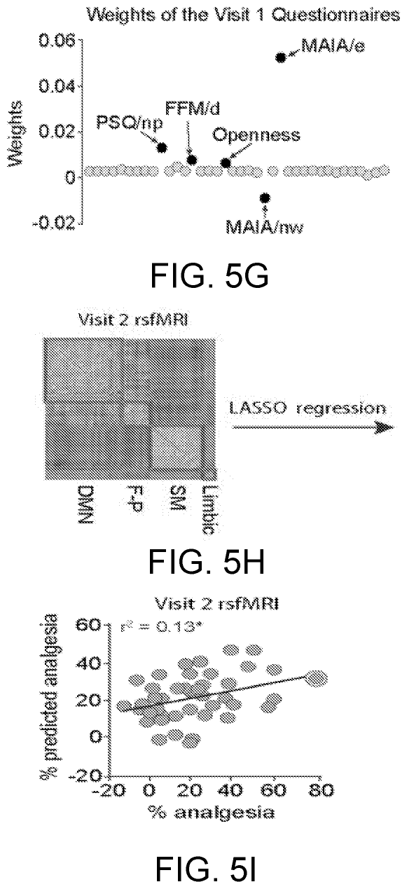

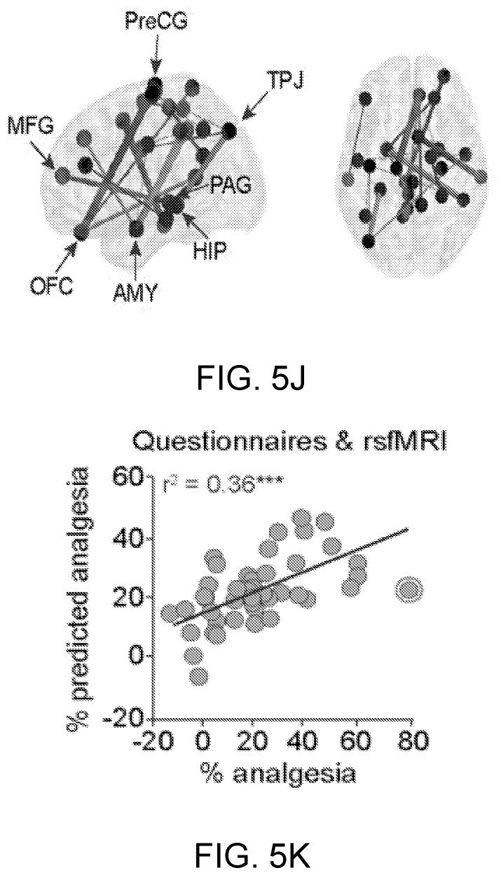

FIGS. 5A-5K illustrates machine learning for predicting the magnitude of response from prospective data, in accordance with an embodiment of the present disclosure. FIG. 5A illustrates that the support vector machine (SVM) classifier is applied to the questionnaire data in a nested leave-one-out cross-validation (LOOCV) procedure. FIG. 5B illustrates that the observed accuracy of this classification analysis is displayed against the null distribution generated using scramble codes from 100 randomized labels. FIGS. 4C-4D illustrate that the classification performance is assessed based on the confusion matrix and the ROC curve shows specificity and sensitivity of the model. FIGS. 4E-4F illustrate that shown the LASSO regression is applied to the questionnaires data and predicted the magnitude of response in the left-out patient. FIG. 5G illustrates that the averaged weights across the n=43 loops of the cross-validation procedure show that five psychological factors predicted the magnitude of response. FIGS. 4H-41 illustrate that the features selected from rsfMRI within each training sample predicted the magnitude of response in the left out patient. FIG. 5J illustrates that a single set of consensus weights is generated by averaging the weight of each feature across the n=43 loops; this consensus set is projected back onto the brain for display. FIG. 5K illustrates that the linear regression analysis indicated that the predicted magnitude of response from rsfMRI at V2 and from personality at V1 are independent contributors that jointly explained 36% of the variance in actual response to placebo treatment. The red circle marks the predicted magnitude of response for the outlier, which is excluded for assessing the error of the model.

### Example II

Validation of a Biosignature for Placebo Response in Chronic Pain.

A biosignature predicting placebo pill response was validated in the settings of a blinded clinical trial in the following example. Chronic pain patients were classified, based on personality and prefrontal cortex functional connections, into predicted-placebo-responders (predicted-PTxResp) and non-responders (predicted-PTxNonR), then randomized into: no-, placebo, or naproxen treatments. Clinically meaningful improvement was higher in the predicted-PTxResp compared to the predicted-PTxNonR. The active drug effects could be isolated using predicted-PTxNonR showing minimal placebo contamination.

As previously noted, the placebo pill effect represents an improvement of symptoms after consuming an inert treatment. In randomized controlled trials (RCTs), patients receiving placebo treatments have shown consistent improvements, especially in the realm of mental health and pain. [1, 2, 3]. However, the mere existence of the placebo effect in RCTs has been questioned, as patients receiving no treatment improved at a similar rate. [4]. Thus, a Cochrane systematic review comparing placebo to no treatment concluded that placebo effects on pain were small and depended on the settings of the RCT, implying that the placebo response is a statistical artifact rather than an actual response to the inert treatment. [5]. There is currently no evidence that placebo pill analgesia is a true biological phenomenon that can be harnessed in the settings of a RCT.

Identifying placebo responders and removing them from RCTs would be a crucial advance, because doing so would reveal the true pharmacological effect of a tested drug and can reduce cost and improve specificity of clinical trial outcomes. Recent studies suggest that pre-existing brain properties determine placebo pill response in fibromyalgia, osteoarthritis (OA) and chronic back pain (CBP) patients. [6, 7, 8, 9, 10]. Yet, the diagnostic value of these brain features for predicting placebo response remain unknown because they have never been validated in an unbiased manner in a new group of patients. Therefore, there are currently no validated biomarkers/biosignatures that can predict placebo response in a clinical trial. In the present study, a multi-dimensional biosignature of placebo response was developed and stratified a new group of patients as responders or non-responders. [10]. The results demonstrate that placebo response was predicted above chance level, implying that placebo response may be harnessed in clinical trials, and potentially also used in individualized treatments for diseases and chronic pain.

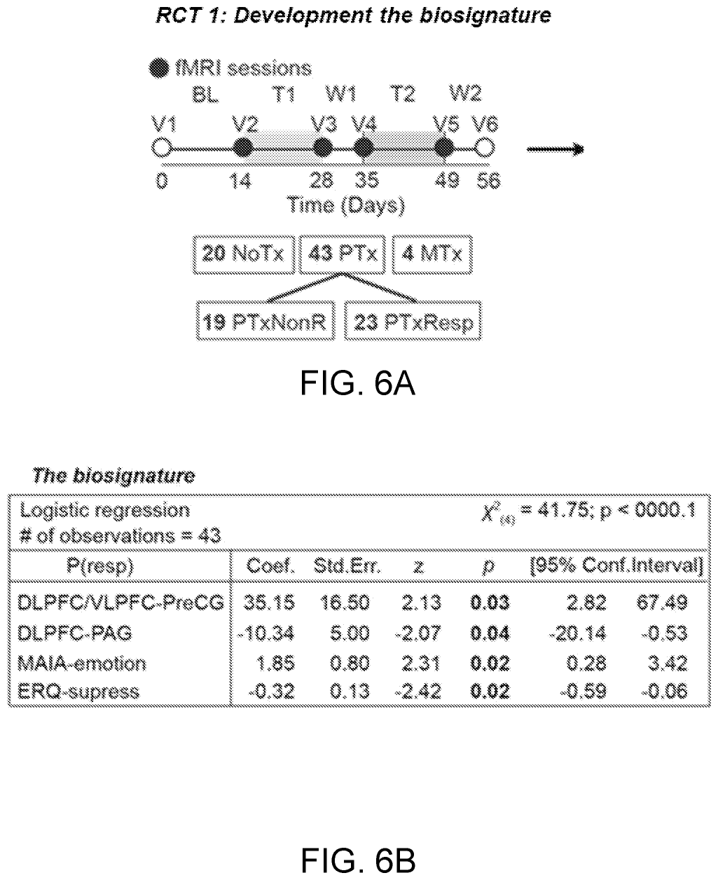

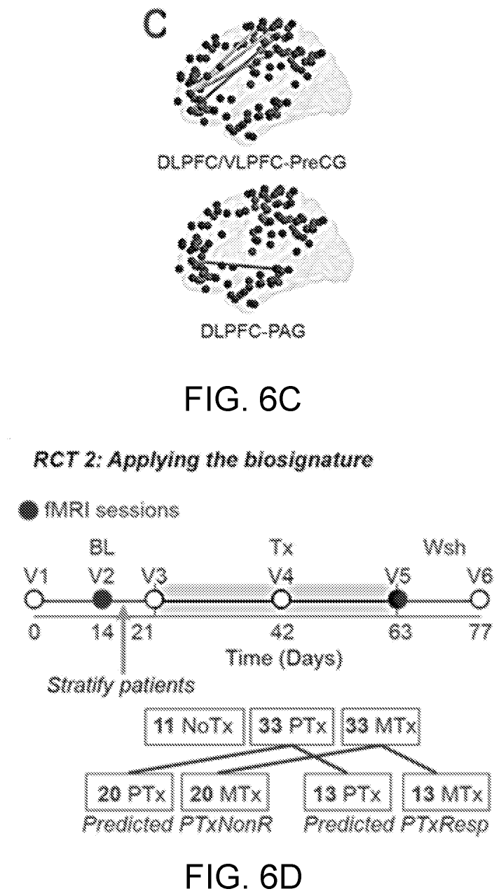

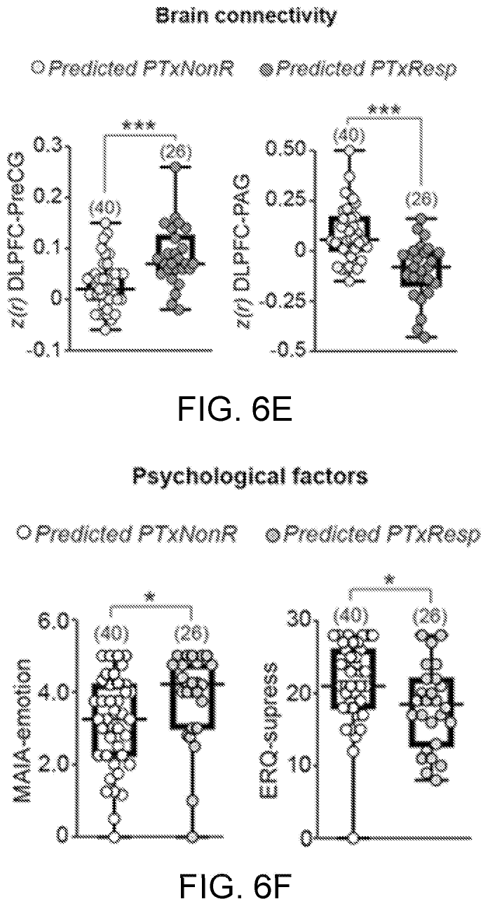

This neuroimaging-based example is the second phase of the research program aiming at predicting placebo response in CBP patients in the setting of a randomized controlled clinical trial. As described in Example I, the psychological and brain features predicting placebo response was derived in a group of CBP patients, by contrasting between 19 placebo responders (PTxResp) and 23 non-responders (PTxNonR) and relative to 20 no-treatment (NoTx) group regarding brain anatomy, function, and personality (see FIG. 6A). (10). A biosignature for placebo response was constructed from four features identified using a logistic regression (FIGS. 6B-C). In the current example (FIG. 6D), the biosignature was applied blindly to these patients using an automated pipeline extracting the features of resting state functional connectivity (FIG. 6E) and combining these with the questionnaire scores (FIG. 6F). The patients were classified as predicted placebo responders (predicted-PTxResp) or non-responders (predicted-PTxNonR) prior to randomization, preventing bias in the validation from model flexibility. Thus, two independent RCTs were used to develop a biosignature in Example 1 and validate the biosignature in the Example 2.

As depicted in FIG. 6A-C, derived biosignature was based on four features: functional connectivity between the dorsolateral prefrontal cortex (DLPFC) and the ventrolateral prefrontal cortex (VLPFC) with the precentral gyrus (Pre-CG; displayed in FIG. 6C) and the periaqueductal grey (PAG; displayed in FIG. 6C); and capacities of awareness (ERQ-suppress) and regulation of emotions (MAIA-emotion). FIG. 6D depicts the Example II study design consisting of six visits to the lab and about 10 weeks duration during which patients were exposed to NoTx, PTx, or MTx lasting 6 weeks. The biosignature from Example I was used to stratify patients as a predicted-PTxResp or predicted-PTxNonR before randomization. As shown in FIG. 6E-F, all four components of the biosignature distinguished between predicted-PTxResp and predicted-PTxNonR in patients randomized in PTx and MTx. * p<0.05, ***p<0.001.

In the current example, 182 CBP patients were assessed for eligibility, from which 77 completed the RCT and were analyzed (FIG. 6D). Once classified, the patients were randomized to: a placebo treatment arm (PTx, n=33); an active medication treatment arm (MTx), a nonsteroidal anti-inflammatory drug, naproxen (n=33); or a no treatment arm (NoTx, n=11), which controls for spontaneous improvement of symptoms contingent on exposure to the healthcare setting. The patients then started a six weeks treatment period at visit 3 (visit 3-visit 5), followed by a two weeks washout period (visit 5-visit 6). Throughout the trial, pain intensity was measured using ecological momentary assessments (EMAs), entered twice a day using a smartphone application, as in Example I.

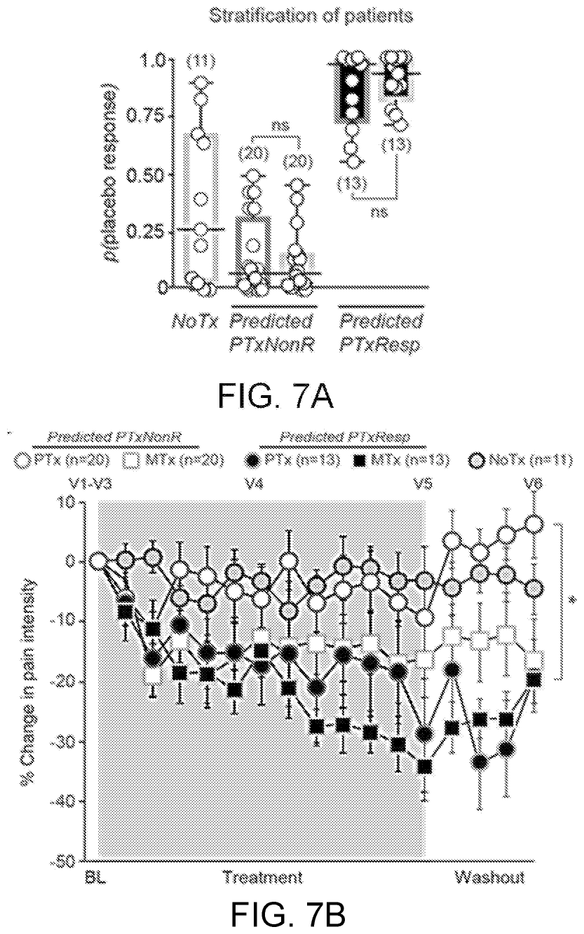

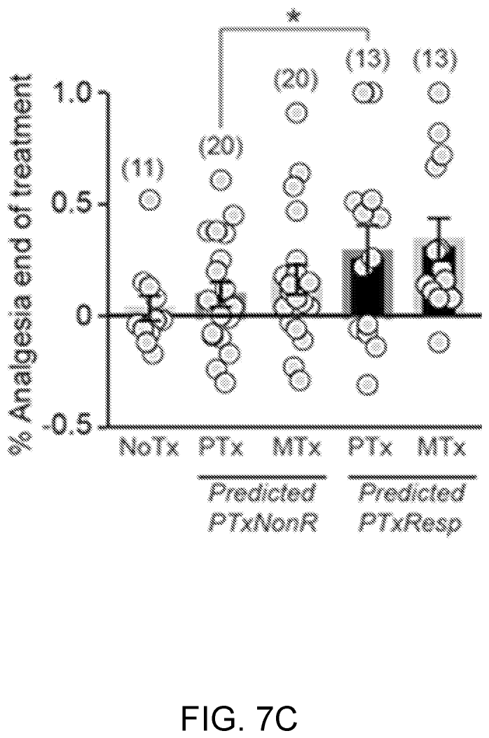

The probability of placebo response is displayed for each patient enrolled in each treatment arm (FIG. 7A). The distribution of probability of placebo response was bimodal, as the median probability of placebo response within each group was closer to either 0 or 1 (FIG. 7A). The biomarker predicted that 30/77 (39%) of the participants will respond to placebo. These were randomized in NoTx (n=4; 36%), PTx (n=13; 39%), and MTx (n=13; 39%). The probability of placebo response was equivalent between patients receiving PTx compared to MTx. The black line shows the median for each group. The time course of pain ratings were different between the five groups (FIG. 7B). Each bin represent averaged EMAs over 1/2 week, as in Phase 1. Planned post-hoc comparisons revealed that predicted PTxResp receiving PTx showed stronger analgesia compared with predicted PTxNonR receiving PTx. As shown in FIG. 7C, the magnitude of % analgesia measured at the last bin of the treatment period was also different between these groups. * planned post hoc comparison p<0.05 one tail

The time course of pain intensity was different between the groups matching the expected trajectories, as displayed in FIG. 7B (F_{(29.5,530.5)}=2.03; p=0.001, repeated measure ANOVA (time*group)). A post hoc planned comparison revealed stronger analgesia in predicted PTxResp compared to predicted PTxNonR receiving placebo pills (Mean difference: 14.9%; p=0.024, one tail). The magnitude of the % analgesia measured at the end of the treatment period was also marginally different between the five groups (F_{(4,72)}=2.41; p=0.057). Post-hoc planned comparison revealed higher analgesia in predicted PTxResp compared to predicted PTxNonR receiving placebo pills (Mean difference: 19.5%; p=0.038, one tail; FIG. 7C.

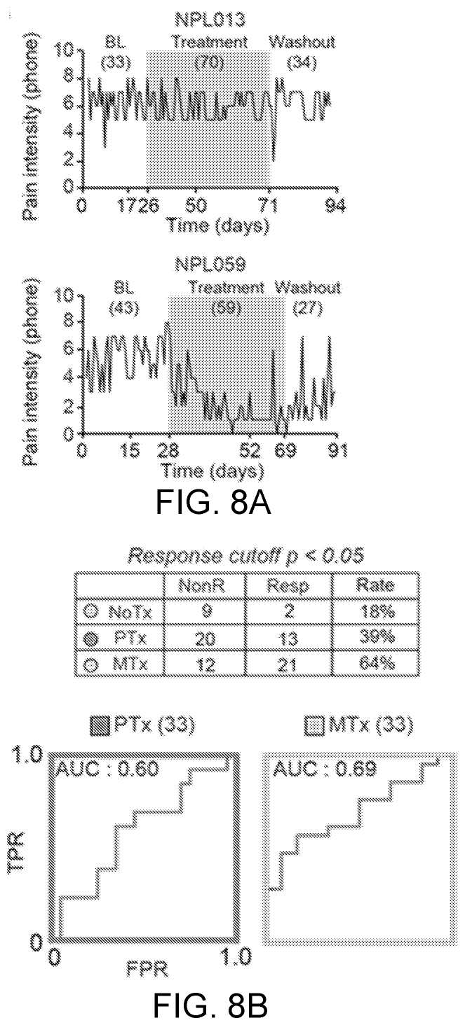

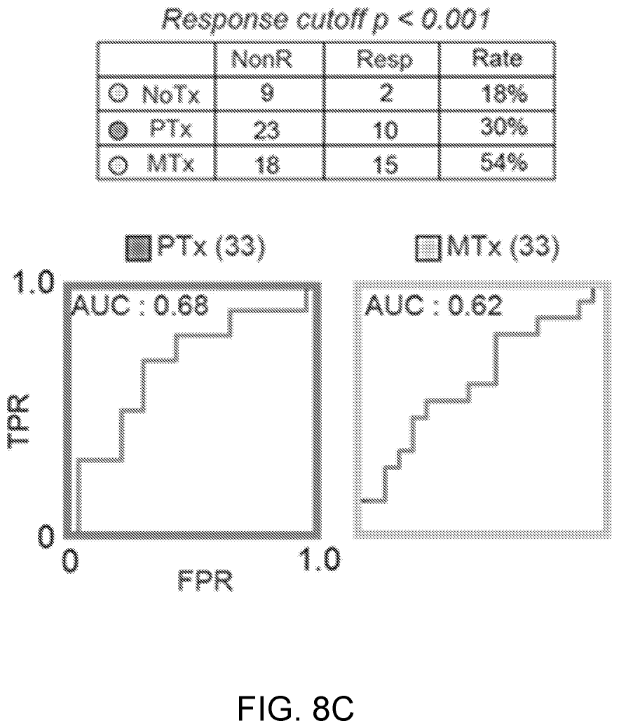

Next, the analgesia specifically attributed to the drug was measured by interrogating the predicted PTxNonR. Assuming that these patients show no placebo contamination, the % change in pain intensity was subtracted in patients receiving placebo pills (FIG. 8E), leaving around 15% residual analgesia specific to the MTx. Group differences in analgesia were detectable with n=40 predicted PTxNonR patients (F_{(1,30)}=3.00; p=0.047, repeated measure ANOVA, one-tail). In comparison, including all 66 patients was sufficient only to detect a trending statistical difference between the MTx and the PTx arms (F_{(1,64)}=1.92; p=0.09, repeated measure ANOVA, one-tail). The results suggest that the biosignature/biomarker of placebo response may theoretically be used in the context of clinical trials, to pre-select placebo non-responders, and detect drug effects with fewer subjects.

The diagnostic value of the biosignature was tested to predict individual response to treatment. It was reasoned that a biosignature of placebo response should predict clinical improvement to both placebo pills and to drug pills (considering the effect attributed to placebo). The patients were classified as responders (Resp) or non-responders (NonR) based on a within subject pain diminution after the start of treatment (FIG. 8A). Within patient clinical improvement was determined using a permutation test comparing pain entries during the 2 weeks baseline and the pain entries during the 6 weeks treatment. The response rate was different across the treatment arms (cutoff of p<0.05, as in phase 1: χ²₍₂₎=8.10; p=0.017; FIG. 8B). The receiver operating characteristic (ROC) curves show the diagnostic ability of the biosignature to predict placebo response for PTx and MTx. The area under the curve (AUC) varied between 0.60 and 0.70, indicating that the model could partly predict the placebo response in both treatment arms (FIG. 8B). Varying the cutoff point defining Resp to p<0.001 yielded similar results (FIG. 8D). Although several patients were misclassified, the biosignature was capable of partly predicting placebo response at the individual level.

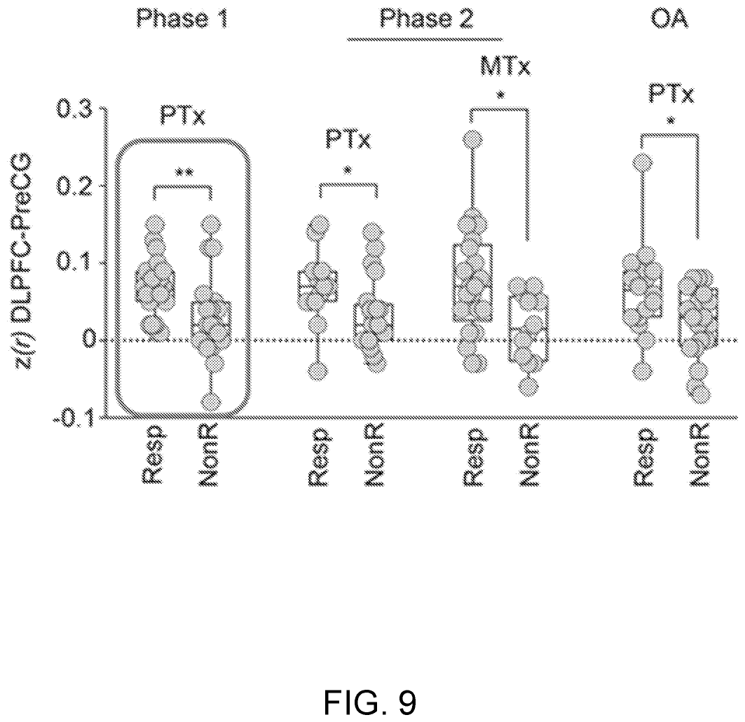

Each feature of the biomarker was examined separately and tested which one segregated Resp from NonR in the current example. There were no group differences between Resp and NonR in psychological factors (all ps>0.22, two-tails) or DLPFC-PAG connectivity (all ps>0.08, two-tails). The functional coupling of the DLPFC-PreCG was however stronger in Resp in both PTx (t₍₃₁₎=2.10, p=0.048, two-tails) and MTx (t₍₃₁₎=2.62, p=0.01, two-tails; FIG. 9). This results was further generalized in a reanalysis of placebo response in OA patients, where Resp also showed stronger DLPFC-PreCG connectivity compared to NonR (t₍₃₄₎=2.23, p=0.03, two-tails). (7).

The findings provide the first evidence for a biosignature of placebo response in the settings of a RCT, which has several important implications. First, it suggests that placebo response in clinical trial can be partially predicted using a biomarker, implying that the phenomenon is not uncontrollable noise. Second, it stresses the role of the DLPFC-PreCG in the response to placebo treatment, across three different RCTs. Third, it has important clinical implications because pre-selecting placebo non-responders can reduce the cost of clinical trials and improve drug development. This exciting finding should, however, be replicated by others, as the sample size of this validation study remains fairly small. Further, the disclosed biosignature was mainly successful based on the DLPFC-PreCG connectivity and retraining new markers on aggregated data across multiple clinical trials may provide more accurate predictions.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

The following references identified by number in the foregoing section are hereby incorporated by reference in their entireties:
1. Kirsch, I. et al. Initial Severity and Antidepressant Benefits: A Meta-Analysis of Data Submitted to the Food and Drug Administration. PLOS Medicine 5, e45 (2008).
2. Kaptchuk, T. J. et al. Components of placebo effect: randomised controlled trial in patients with irritable bowel syndrome. BMJ 336, 999-1003 (2008).
3. Haake, M. et al. German Acupuncture Trials (Gerac) For Chronic Low Back Pain: Randomized, Multicenter, Blinded, Parallel-Group Trial With 3 Groups. Arch Intern Med 167, 1892-1898 (2007).
4. Hróbjartsson, A. & Gøtzsche, P. C. Is the Placebo Powerless? New England Journal of Medicine 344, 1594-1602 (2001).
5. Hróbjartsson, A. & Gøtzsche, P. C. Placebo interventions for all clinical conditions. Cochrane Database Syst Rev CD003974 (2010). doi:10.1002/14651858.CD003974.pub3
6. Schmidt-Wilcke, T. et al. Resting state connectivity correlates with drug and placebo response in fibromyalgia patients. Neuroimage Clin 6, 252-261 (2014).
7. Tetreault, P. et al. Brain Connectivity Predicts Placebo Response across Chronic Pain Clinical Trials. PLoS Biol 14, e1002570 (2016).
8. Hashmi, J. A. et al. Functional network architecture predicts psychologically mediated analgesia related to treatment in chronic knee pain patients. J. Neurosci. 34, 3924-3936 (2014).
9. Hashmi, J. A. et al. Brain networks predicting placebo analgesia in a clinical trial for chronic back pain. Pain 153, 2393-402 (2012).
10. Vachon-Presseau, E. et al. Brain and psychological determinants of placebo pill response in chronic pain patients. Nature Communications 9, 3397 (2018).

1. A method for identifying a placebo responder in an individual or a group of individuals comprising:
obtaining an individual or group of individuals as participants;
pre-specifying inclusion criteria;
conducting a study, wherein the study is conducted in a clinical randomized controlled trial setting for assessing the placebo response, and wherein the study is conducted across at least six visits to the randomized controlled setting;
randomizing participant allocation pertaining to the study, wherein the randomization scheme assigns each participant a randomization code and classifies the participants into one of: placebo group, no treatment group, active treatment group;
administering treatment to the participants;
monitoring pain intensity of the participants by receiving pain ratings from the participants, via a software application, and wherein the software application is executed on a smartphone or a computer connected to the internet;
pre-processing the ratings;
performing a blind analysis;
performing brain imaging and data analysis;
analyzing questionnaire data;
predicting a magnitude of a placebo response; and
administering further treatment to the individual or group of individuals based on brain properties and psychological determinants of the placebo response.

2. The method according to claim 1, wherein the participants meet one or more of the following inclusion criteria:
an age more than or equal to 18 years;
a history of lower back pain for at least 6 months;
have not been prescribed any concomitant pain medications;
have passed the MRI safety screening requirements at each scanning visit; and
have an access to use a smartphone or a computer.

3. The method according to claim 1, where the pain is monitored at least twice a day.

4. The method according to claim 1, wherein the participants are selected from the group consisting of chronic back pain patients, osteoarthritis patients, fibromyalgia, peripheral neuropathy patients, and a combination thereof.

5. The method according to claim 1, wherein the brain properties include subcortical limbic volume asymmetry, sensorimotor cortical thickness, functional coupling of dorsolateral prefrontal cortex (DLPFC) with periaqueductal grey (PAG), rostral anterior cingulate cortex (rACC), and precentral gyrus (PreCG), or a combination thereof.

6. The method according to claim 5, wherein the brain properties are identified and evaluated by the brain imaging.

7. The method according to claim 6, wherein the brain imaging includes a high-resolution T1-weighted brain image, at least one resting state scan, and at least one diffusion tensor imaging scan collected from the individuals using integrated parallel imaging techniques.

8. The method according to claim 1, wherein psychological determinants include optimism, anxiety, extraversion, neuroticism, or a combination thereof.

9. The method according to claim 1, wherein the psychological determinants are identified and evaluated by the questionnaire data.

10. The method according to claim 1, wherein the participants include individuals at least 18 years old with a history of lower back pain for at least 6 months, wherein the lower back pain is neuropathic, with no evidence of additional co-morbid chronic pain, neurological, or psychiatric conditions.

11. The method according to claim 1, wherein the total duration of the study is 2, 4, 6, 12, or 15 months.

12. The method according to claim 1, wherein pain ratings include a baseline and ratings from at least one treatment.

13. The method according to claim 1, wherein the brain properties, the psychological determinants, and the pain ratings are evaluated to identify a placebo responder.

14. A method of treating a patient with chronic pain according to the method of claim 13, and wherein the treatment is inert.

15. A method of identifying a placebo responder in a randomized controlled trial according to claim 13.

16. A method of creating a biosignature for use as a predictor of placebo response comprising:
neuroimaging a brain of an individual with magnetic resonance imaging;
identifying a prefrontal cortex functional connection, wherein the prefrontal cortex functional connection is a functional coupling of a dorsolateral prefrontal cortex (DLPFC), a periaqueductal grey (PAG), a rostral anterior cingulate cortex (rACC), a precentral gyrus (PreCG); or a combination thereof;
identifying capacity of awareness and regulation of emotions, wherein the capacity of awareness and the regulation of emotions is determined by the individual personality traits, wherein the personality traits are determined by the individual completing a questionnaire, and wherein the personality traits include optimism, anxiety, extraversion, neuroticism or a combination thereof; and
stratifying the individual based upon the prefrontal cortex functional connection, capacity of awareness, and regulation of emotions.

17. The method of claim 16, wherein the individual is stratified as a predicted placebo responder or a non-responder.

18. A method of treating a patient with chronic pain using the biosignature of claim 17, and wherein the treatment is inert.

19. A method of identifying a placebo responder in a randomized controlled trial and removing the placebo responder according to the method of claim 17.

20. A method of treating a patient with chronic pain comprising:
neuroimaging a brain of a patient with magnetic resonance imaging;
identifying a prefrontal cortex functional connection, wherein the prefrontal cortex functional connection is a functional coupling of a dorsolateral prefrontal cortex (DLPFC), a periaqueductal grey (PAG), a rostral anterior cingulate cortex (rACC), a precentral gyrus (PreCG); or a combination thereof;
identifying capacity of awareness and regulation of emotions, wherein the capacity of awareness and the regulation of emotions is determined by the individual personality traits, wherein the personality traits are determined by the individual completing a questionnaire, and wherein the personality traits include optimism, anxiety, extraversion, neuroticism or a combination thereof; and
determining if the patient is a placebo responder based upon the prefrontal cortex functional connection, capacity of awareness, and regulation of emotions; and
treating the patient with chronic pain, wherein the treatment is inert, and wherein the chronic pain includes chronic back pain, osteoarthritis, fibromyalgia, or peripheral neuropathy.
